(19) United States
(12) Patent Application Publication
(10) Pub. No.: US 2016/0077091 A1
(43) Pub. Date: Mar. 17, 2016

(54) PORTABLE TESTING SYSTEM FOR DETECTING SELECTED DRUGS OR COMPOUNDS IN NONCONTROLLED ENVIRONMENTS

(71) Applicant: EXPRESS DIAGNOSTICS INTERNATIONAL, INC., Blue Earth, MN (US)

(72) Inventors: Steven Patrick TYRRELL, Erie, CO (US); Barry Patrick VANT-HULL, Boulder, CO (US); Dean Michael KINGSTON, Arvada, CO (US)

(21) Appl. No.: 14/787,254

(22) PCT Filed: Apr. 25, 2014
(86) PCT No.: PCT/US14/35552

§ 371 Date: Oct. 26, 2015

Related U.S. Application Data
(60) Provisional application No. 61/816,433, filed on Apr. 26, 2013.

(51) Int. Cl. G01N 33/543; G01N 33/94

(52) U.S. Cl. CPC G01N 33/54386; G01N 33/94

(57) ABSTRACT

Devices, methods, and systems are provided for testing for or detecting the presence of illegal or prohibited drugs, compounds or metabolites in non-controlled environments for testing using lateral flow drug test strips and a smart phone with digital imaging, data processing, data storage and wireless electronic transmission of resulting data, the system or method to test dry buffer conditioned oral fluids using lateral flow test strips for determination of the presence or absence, or other quantitative or qualitative measurement, of specific and/or selected drugs, compounds or metabolites thereof.

### FIELD OF THE DISCLOSURE

The disclosure relates generally to the field of automated and portable lateral flow drug test strip system, apparatus, and methods for testing individuals for threshold amounts of drugs or compounds, or metabolites thereof.

### BACKGROUND

Lateral flow tests, also known as Lateral Flow Immunochromatographic Assays, are simple devices intended to detect the presence (or absence) of a target drug, compound, or metabolite in sample (matrix) without the need for specialized and costly equipment, though many lab based applications exist that are supported by reading equipment. Typically, these tests are used for medical diagnostics either for home testing, point of care testing, or laboratory use. A widely spread and well known application is the home pregnancy test.

The technology is based on a series of capillary beds, such as pieces of porous paper or sintered polymer. Each of these elements has the capacity to transport fluid (e.g., urine) spontaneously. A first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the drug, compound, or metabolite binds to the particles while migrating further through the third capillary bed. This material has one or more areas (often called stripes) where a third molecule has been immobilized by the manufacturer. By the time the sample-conjugate mix reaches these stripes, drug, compound, or metabolite has been bound on the particle and the third ‘capture’ molecule binds the complex. After a while, when more and more fluid has passed the stripes, particles accumulate and the stripe-area changes color. Typically there are at least two stripes: one (the control) that captures any particle and thereby shows that reaction conditions and technology worked fine, the second contains a specific capture molecule and only captures those particles onto which a drug, compound, or metabolite molecule has been immobilized. After passing these reaction zones, the fluid enters the final porous material, the wick, that simply acts as a waste container. Lateral Flow Tests can operate as either competitive or sandwich assays.

Numerous systems and instruments have been created to aid in the detection of illegal drugs or compounds in persons suspected of imbibing, ingesting, injecting, or inhaling illegal or controlled drugs or compounds. These systems have been created for use in the fields of law enforcement testing systems. These systems range from handheld portable devices such as blood alcohol testers to breathalyzers Despite this evolution in testing, the majority of these systems suffer from many problems, such as, but not limited to, the lack of usefulness by law enforcement as a portable, self-calibrating, and automated system that provides automated or easy setup, calibration, sample collection and application, reading, analysis, and result generation, reporting, and communications. Additionally such known systems lack portable easy, efficient or accurate body fluid sample preparation, such as accurate, fast, and easy saliva buffering or conditioning, as well as the lack of an integrated panel display or GUI interface that in a portable and easily used system for roadside use by law enforcement that generates accurate readings and results that are recorded and automatically reported or communicated to law enforcement or the court systems. These limitations often result from building systems using application specific devices that lack hardware, software and processing flexibility to address suitable test architectures and protocols that can be easily and accurately used by law enforcement and others. As a result, existing systems fail to provide an easily used and accurate, but portable and roadside suitable, system that the average law enforcement officer can use, that is also cost effective and suitable for all types of illegal or controlled drug or compound testing.

Accordingly, there is a need for a portable, roadside illegal or controlled drug testing system that overcomes one or more of these problems.

### SUMMARY

Devices, methods, and systems are provided for testing for or detecting the presence of illegal or prohibited drugs, compounds or metabolites in non-controlled environments for testing using lateral flow drug test strips and a smart phone with digital imaging, data processing, data storage and wireless electronic transmission of resulting data, the system or method to test dry or other buffer conditioned oral fluids using lateral flow test strips for determination of the presence or absence, or other quantitative or qualitative measurement, of specific and/or selected drugs, compounds or metabolites thereof.

Non limiting optional embodiments can include devices, methods, software, computer readable computer systems, and/or systems for testing for or detecting the presence of drugs or other chemicals or metabolites, e.g., but not limited to, illegal or prohibited drugs, in non-controlled or difficult to control environments. A system can use oral fluid, saliva, and/or other tissue or bodily fluids (such as blood or plasma) as the test sample. Non limiting optional embodiments can optionally include one or more of an electronic device such as a smart phone or other wireless, internet or cellular communications capable device, in combination with digital and/or other imaging, data processing, data storage and/or wireless electronic transmission of data via cellular networks or Wi-Fi. Non limiting optional embodiments also can include a case, housing, or other container or carrier, optionally including lighting and/or positioning. A system or method can provide one or more of collect, detect, process, manipulate, alter, condition, determine, validate, and/or test oral or other bodily fluids or tissues in volumes sufficient for testing, chemically and/or mechanically collecting, detecting, processing, manipulating, altering, conditioning, determining, validating, and/or testing the oral or other bodily fluid or tissue. A system or method can further provide for the set up and/or optimize for testing and/or delivering the conditioned oral fluids to lateral flow test strips or other testing systems for determination of the presence or absence, or other quantitative or qualitative measurement, of specific and/or selected drugs, chemicals or biological materials, such as illegal or prohibited drugs or compounds.

Non limiting optional embodiments can optionally include testing and/or reader devices that can be, e.g., but not limited to, a purpose dedicated or other smart phone or other device used for capturing digital images to be used for collecting, analyzing, qualifying, quantifying, processing, validating, determining, and/or verifying test results, collecting relevant identification from the person being tested, storing this information as a secure data file and/or wirelessly transmitting the information to specified law enforcement, court of jurisdiction, district attorney, or other authorities and/or related secure or encrypted databases or servers. Such devices can also optionally provide for timing the test endpoint and/or subsequent image capture by either automatic initiation of timing once the testing device is inserted into the positioning case of by the user initiating the start time of the test by interacting with a touch sensitive or other graphical user interface. The timing can be test specific by the electronic device capturing specific information printed onto the testing device immediately after insertion into the postponing case or by the user entering test identification information into the electronic device.

The present disclosure is optionally directed, in general, to a portable lateral flow drug test strip system for testing the presence or amount of a drug, compound, or metabolite thereof, comprising:
an exterior protective case;
a test cartridge receptor provided within said case to receive a test cartridge having one or more reading bays configured to hold pre-calibrated lateral flow drug test strips inserted therein, said test cartridge or test strip comprising or provided with soluble or dry buffers for preparing a saliva sample from a person being drug tested that is applied to said one or more drug test strips,
a housing provided with or in the protective case for positioning digital camera hardware, illumination hardware, a computer processor, data memory storage, and wireless communications, the digital camera hardware comprising imaging optics to produce a digital image of one or more of said test strips in said test cartridge and related testing and identification images and data;
software provided in said data memory storage and loadable onto said computer processor, said software calibrating, recording, capturing, and analyzing said test strip images to provide drug testing results that are stored in said data memory storage along with ID images and data identifying the person for which saliva is collected from, said results, ID images and data transmissible via said wireless communications to a remote server or communications device; and
an optional graphical user interface (GUI) for providing tester controlling and using the system for electronically collecting, entering, calibrating, analyzing, and transmitting information, images, data, or results, relating to the drug test or the persons being tested or conducting the test; including electronically providing instructions via said GUI to set up and operate said system; and
an optional location detection module, wherein the location detection module can be a global positioning system (GPS) transceiver, any known location determining system, or a network location provider which provides location based on signal strengths from WiFi hotspots or cell towers with known or determinable locations;
wherein said drug test strip or a casing thereof when provided with the conditioned sample indicates the presence or amount of a drug, compound, or metabolite thereof in said saliva to determine whether said amount is above a pre-selected threshold or concentration. A portable lateral flow drug test strip system can optionally provide wherein the system comprises a smart phone comprising three or more of said: digital camera, illumination hardware, computer processor, data memory storage, wireless communications, imaging optics, software, GUI, and location detection module.

A portable lateral flow drug test strip system can optionally provide wherein the drug test image indicates the presence or amount of drug specific to each drug test strip.

A portable lateral flow drug test strip system can optionally provide wherein the system further comprises a heater to maintain said drug test strips within specified temperature ranges suitable for said drug test.

A portable lateral flow drug test strip system can optionally provide wherein said reading bays comprise at least a first type of reading bay having a first width and a first depth and the second type of reading bay has a second width and a second depth, and wherein each of said reading bays has a single opening to receive each of the first type of test cartridge and the second type of test cartridge without the use of a separate adapter.

A portable lateral flow drug test strip system can optionally provide wherein the test cartridge receptor includes an internal track guide for positioned insertion of test cartridges sized or calibrated to pre-position said test strips relative to said digital camera to size the digital image for said electronically collecting, entering, calibrating, analyzing, and transmitting information, images, data, or results, relating to the drug test.

A portable lateral flow drug test strip system can optionally provide wherein the computer and/or software comprises a test cartridge or test strip detection and identification logic to detect and identify the presence of a particular test cartridge or test strip to determine the type of drug test to provide said software calibrating, recording, capturing, and analyzing said test strip images to provide drug testing results that are stored in said data memory storage.

A portable lateral flow drug test strip system can optionally provide wherein the test cartridge receptor has a side, bottom or top insertion track or alignment for receiving said test cartridges.

A portable lateral flow drug test strip system can optionally provide wherein the location detection module provides location data that is communicated to the computer processor or memory data storage to record the location of the drug test performed using said portable and/or wireless based lateral flow drug test strip system.

A portable lateral flow drug test strip system can optionally provide wherein the system further comprises an environmental sensor to detect environmental conditions.

A portable lateral flow drug test strip system can optionally provide wherein the environmental conditions are selected from humidity and temperature data, airborne contaminants, and toxins.

A portable lateral flow drug test strip system can optionally provide wherein said pre-selected threshold is the legal limit for said drug, compound, or metabolite thereof, or said preselected concentration is the legal or illegal concentration of said drug, compound, or metabolite thereof, for the corresponding legal jurisdiction.

A portable lateral flow drug test strip system can optionally provide wherein said drug or compound is optionally selected from alcohol, cocaine, methamphetamine, heroin, THC, PCP, psilocybin, opiate drugs, or other know drugs or illegal or controlled substances or derivatives thereof, e.g., as known in the art or as listed or described herein. According to non-limiting optional embodiments, the drug test strip system and method of Non limiting optional embodiments can be used to test for drugs, compounds, derivatives thereof, or plants or fungi from which they are derived from, which can include, but are not limited to, amphetamines (Amphetamine, dextroamphetamine and methamphetamine), alcohol, anabolic steroids, anorectic drugs (e.g., benzphetamine (Didrex®), diethylproprion (Tenuate®, Tepanil®), mazindol (Sanorex®, Mazanor®), phendimetrazine (Bontril®, Prelu-27®), and phentermine (Lonamin®, Fastin®, Adipex®), barbiturates (e.g., methohexital (Brevital®), thiamyl (Surital®) and thiopental (Pentothal®); amobarbital (Amyta®), pentobarbital (Nembutal®), secobarbital (Seconal®), Tuinal (amobarbital/secobarbital combination); butalbital (Fiorina®), butabarbital (Butisol®), talbutal (Lotusate®), and aprobarbital (Alurate®)); benzodiazepines (estazolam (ProSom®), flurazepam (Dalmane®), temazepam (Restoril®), triazolam (Halcion®); Midazolam (Versed®), alprazolam (Xanax®), chlordiazepoxide (Librium®), clorazepate (Tranxene®), diazepam (Valium®, halazepam (Paxipam®), lorzepam (Ativan®), oxazepam (Serax®), prazepam (Centrax®), and quazepam (Doral®); clonazepam (Klonopin®), clorazepate; Flunitrazepam (Rohypnol®), Zolpidem (Ambien®) and zaleplon (Sonata®)); butorphanol, buprenorphin, bufotenine, Cannibis (THC, marijuana, hashish, hash oil, hemp.), chloral hydrate, coca leaf, cocaine, codeine, cocaine, depressants, dextropropoxyphen, DET, DOB, DOM, DXM, Ecstasy (MDMA), ephedra, fentanyl, flunitrazepam, foxy, GBL, GHB, glutethimide, hallucinogens (LSD,), Opiates (heroin,); hydrocodone, hydromorphone, ketamine, K2 spice, khat, LAAM, magic mushrooms (e.g., AET, psilocybin, psilocin, peyote), MDA, meperidine, meprobamate, mescaline, methadone, methamphetamine, methcathinone, methaqualone, meth labs, methylphenidate, morphine, narcotics, NEXUS, opium, opium poppy, oxycodone, oxycontin, paraldehyde, PCP, pentazocine, peyote, prescription drugs, ritalin, rohypnol, salvia divinorum, san pedro cacti, STP, thebaine, tryptamines, 1,4 butane diol, 2C-B, 5MeO-AMT, or derivatives or synthetic derivatives thereof.

A portable lateral flow drug test strip system can optionally provide wherein the person performing the test (tester) performs at least one selected from the group consisting of removing the test device from the packaging, removing a cap from an oral fluid collecting device or placing the oral fluid collecting device in a test subject's mouth.

A portable lateral flow drug test strip system can optionally provide wherein the tester:
opens the case on the electronic device and/or via the GUI in order to initiate the drug test;
collects oral fluid until sufficient oral fluid has been collected (absorbed) by the test device as indicated by a specified color changing fill indicator or the tester removes from the mouth of the test subject when complete or replaces the cap; and/or
removes a test initiation sticker or inserts the collected oral fluid into test cartridge in the system.

A portable lateral flow drug test strip system can optionally provide wherein the test timing is automatically initiated by insertion of collected oral fluid, or the tester initiates test timing on the GUI of the device after insertion.

A portable lateral flow drug test strip system can optionally provide wherein the digital camera takes an initial picture of the test device to determine the specific test type information or lot numbers or any other specific information.

A portable lateral flow drug test strip system can optionally provide wherein the case initiates internal lighting automatically to illuminate the test strips when an image is being captured by the digital camera.

A portable lateral flow drug test strip system can optionally provide wherein test or control line intensities on each test strip tested are electronically determined from the digital image by said system.

A portable lateral flow drug test strip system can optionally provide wherein the positive or negative result, or validity, of the test is electronically determined by analyzing said control line intensities for each test strip.

A portable lateral flow drug test strip system can optionally provide wherein the results of the test are electronically communicated to at least one of a tester, law enforcement personnel, or a court.

### BRIEF DESCRIPTION OF THE DRAWINGS

FIG. 1 depicts a schematic representation of a system of non-limiting optional embodiments.

FIG. 2 depicts an exemplary embodiment of a lateral flow drug test system of non-limiting optional embodiments.

FIG. 3 depicts exemplary embodiments of test strips.

FIG. 4A-B depicts an exemplary method of the system level testing sequences.

FIG. 5 depicts an exemplary method of an algorithm based digital image test strip analysis.

FIG. 6A-6C depicts alternative views of an exemplary embodiment of Example 1 of a test strip and casing and how it is arranged, manufactured and/or used in non-limiting optional embodiments.

FIG. 7 depicts an exemplary embodiment of Example 2 showing how a mobile app can be used according to non-limiting optional embodiments to conduct drug testing using a device and/or method.

FIG. 8A-8B depicts alternative views of exemplary embodiment of Example 3 of a test strip and casing and how it is arranged, manufactured and/or used in non-limiting optional embodiments.

A use of the same reference symbols in different drawings indicates similar or identical items.

### DESCRIPTION

A present disclosure is directed, in general, to a portable roadside and/or wireless based lateral flow drug test strip systems, devices, or methods, saliva or body fluid conditioning lateral flow test strip systems, devices or methods, optional testing cartridges, systems, and methods, and a method of coupling lateral flow drug test strip systems with test cartridges, a protective case and/or housing, digital camera hardware, computer processor, memory data storage, and software for calibrating, recording, capturing, and analyzing said test strip images to provide drug testing results that are stored in the data memory storage along with ID images and data identifying the person for which saliva or body fluid is collected from, for said drug test, and transmissible via the wireless communications to a remote server or communications device; and/or
an optional graphical user interface (GUI) for setting up and conducting the drug testing using the system and for electronically collecting, entering, calibrating, analyzing, and transmitting information, images, data, or results, relating to the drug test or the persons being tested or conducting the test; including electronically providing instructions via said GUI to set up and operate said system; and/or
an optional location detection module, wherein the location detection module is a global positioning system (GPS) transceiver; and/or
wherein said drug test strip optionally analyzes the presence or amount of a drug, compound, or metabolite thereof in said saliva to determine whether said amount is above a pre-selected threshold or concentration.

Non limiting optional embodiments can optionally include one or more of the following types of functionalities and/or controls:
1. Pre-Read Calibration: A device, method or system can optionally do one or more of the following, as non-limiting examples: determine whether the illumination hardware and/or the camera are both functioning correctly; whether the test card has been inserted correctly; analyzing control strips or color swatches placed in the reading bay for calibration to determine if the lighting and/or camera is working properly or malfunctioning; analyzing the image at the edges of where the test card is expected to be to determine whether insertion is correct.
2. Image Capture And/or Analysis: A device, method or system can optionally do one or more of the following, as non-limiting examples: redefined or specified or calibrated regions of the image can optionally be recorded or extracted, digitized, and/or or recorded for analysis; e.g., but not limited to, the top of each drug, compound, or metabolite or test strip or casing or holder can be color or other coded to specify, label and/or identify the analyte. Indicator lines are expected in well-defined regions of the test strip or casing or holder. Intensities or values of these lines can optionally be determined from these regions.
3. Specific Drug, compound, or metabolite Analysis: A device, method or system can optionally do one or more of the following, as non-limiting examples: color coding can be matched to a lookup table to determine which drug, compound, or metabolite is being measured on each individual strip. A software can include library algorithms for each analyte, used to determine whether a result is positive or negative based on the relative intensities of the control line and/or test line.
4. Capture of Collateral Images: A device, method or system can optionally do one or more of the following, as non-limiting examples: where pictures of an ID of, or of the individual being tested for inclusion with the results file; for instance, frontal and/or profile pictures with a picture of any relevant identification.
5. Transmission Of Results: A device, method or system can optionally do one or more of the following, as non-limiting examples: file and/or collateral images: smart phones (e.g., Android™, and/or the like) by including the ability to send data by text message or email. Such a message could include the results, the image from which the results were derived, and/or any collateral images. A recipient could be pre-specified, or chosen from a contact list.

Non limiting optional embodiments can optionally further include wherein the electronic device case provides for desired or optimal lighting and/or test device positioning for image capture and/or electronic device protection from mechanical damage and/or optional thermal control for cold environments.

Non limiting optional embodiments can further include wherein the electronic device can be at least partially or fully enclosed in a case that serves to provide for optimal positioning of the testing device in front of a photographic or digital camera that is included or can be provided with the electronic device and/or optionally providing optimal lighting for image capture. This case can fold flat and/or against the electronic device when not in use to make the assembly easily portable in a variety of settings. An optional added feature of Non limiting optional embodiments can include the use in cold environments by providing a heat generating region that the test device that can be in contact with before or during the test process in order to allow for the device or system to be used in cold environments that could affect test results in order to correct for temperature effects on the testing process.

Optional Functionality of systems, devices, or methods of the non-limiting optional embodiments can include one or more of:
1. When not in use, the case can optionally close or fold flat against the electronic device making transport and/or storage easy, e.g., but not limited to, when closed, the case does not increase the geometric volume of the electronic device by more than 60%. A lighting for digital image capture can optionally be powered by electronic device, battery, and/or power source, and which can optionally be controlled by device CPU and/or by insertion and/or removal of test device; or, e.g., internally powered by battery and/or controlled by insertion of the test device.
2. Optionally, in order to help ensure that the test device is correctly positioned under the digital camera, a case can optionally have a slot that when the case is open, the test device can be inserted into the slot and/or the fixturing on the inside of the case can ensure the proper positioning of the device under the camera lens. This positioning can be for one or more of distance from the lens and/or positioning within the frame of the digital image. A test device can also optionally be, or preferably be, in the proper position with respect to the illumination system provided within or associated the case.
3. This system can optionally be used in cold or hot environments, where lateral flow test results can be affected due to low or high temperatures. Lateral flow tests can optionally tolerate large operating temperature ranges so tight temperature control may not be necessary, e.g., approximately 17° C. up to 35° C., would optionally be adequate to ensure accurate results. However, a thermal control mechanism can be used to control temperature when suggested or needed to help ensure accurate test results, e.g., but not limited to, a resistance based or Peltier effect based and/or the temperature measurement may be accomplished digitally or simply have an incubation time for the device residing in the reader case to ensure that the test can occur in the wide functional range cited.

### Exemplary and Optional Features, Elements, Functions, or Aspects of a Testing Device of Non-Limiting Optional Embodiments:

A oral fluid collection and/or testing device can optionally collect oral fluids directly from the mouth of the person being tested by absorption driven by capillary forces native to the specified absorptive materials, the device can optionally have an indicator that changes color or provides an visual, mechanical, or electrical indicator when the device is full of, or has sufficient, oral fluid to conduct the selected test. This indicator can also serve as a passive timer for chemically conditioning the oral fluid since dissolution of the dried chemicals and/or action of these chemicals on the components of saliva is not an instantaneous process and/or can require some time to optimally condition the oral fluid. As opposed to the person supervising the testing conducting or providing the timing the oral fluid conditioning portion of the process, this functionality can optionally be included as a feature of the testing device itself. This timing can be varied, as a non-limiting example, by changing the distance of migration or the materials supporting migration to reach an optimal time for conditioning.

A feature of the testing device, as provided in non-limiting optional embodiments, is that introduction of the conditioned oral fluid to the lateral flow drug test strips can optionally not occur until sufficient incubation time has elapsed and/or a label or sticker is removed from the device. As the device is filling with or collecting oral fluid, the oral fluid can enter a region containing conditioning chemicals and/or buffers. A testing device can optionally contain multiple compartments that can optionally be functionally air tight from the outside environment when fluids are introduced to the device unless a vent is added. In order for fluid to optionally flow into the materials included in each compartment, a vent to the outside can optionally be present to allow air displacement by the entering fluid. If the vent isn't present, fluid may optionally not enter the compartment by capillary forces alone. By placing a removable sticker over the vent of the chamber(s) containing the later flow test strips, the user can optionally control when the fluid enters this chamber. In one embodiment, the testing initiation sticker covering the vent for the test strip chamber can optionally only be removed when the device indicates sufficient oral fluid has been collected and/or because of the preset passive timing aspects that are optionally designed into the fill indicator, when sufficient time has elapsed for the oral fluid to be conditioned, which was in contact with the conditioning chemicals before the fill indicator indicated “full” and/or would then be in an optimal state for lateral flow drug testing.

A oral fluid collection portion of the device can optionally have a region where fluid flow can optionally only be driven forward by capillary forces alone and/or not by pressure of vacuum exerted from the region of the collection device that would reside in the test subjects' mouth. This can optionally be accomplished by having a region of absorbent transport material distal to the portions that can be inside the mouth that is in fluid and/or material connection with further distal absorbent materials but is also open the outside environment providing for a path of least resistance for any air that could be forced into the device to be expelled to the outside due to pressure being exerted from the mouth of the test subject and/or conversely for outside air to be pulled into the portion of the collection device residing in the test subjects mouth should suction be applied preventing tampering and/or migration of any internal device chemicals into the test subjects mouth.

Key optional functions of Non limiting optional embodiments can include one or more of:
1. Collection of oral fluids and/or ensuring that the filling of the internal compartments of the testing device only occurs through capillary forces generated by the liquid interacting with the transport materials incorporated within the device. No test subject generated pressure of vacuum effects.
2. Ensuring that the oral fluid spends a set or minimum time with the oral fluid conditioning chemicals.
3. A fill indicator can optionally serve two purposes.
   a. Indicate that sufficient oral fluid has been collected to ensure that the device has sufficient sample to complete testing.
   b. A time required to fill is optionally not directly related to the time it takes for the indicator to indicate a full state. A observed fill time can be artificially extended to ensure that the oral fluid has incubated with the conditioning chemicals a sufficient amount of time.
      i. This can optionally be accomplished by the choice of materials in the fill indicating area since these can be chosen based on lateral flow rates thus impacting the observed fill time.
      ii. This can also be optionally accomplished by simply lengthening the migration distance of the oral fluid in the fill indicator area to modify the observed fill time.
4. A removable label that can when present prevent the flow of oral fluid onto the later flow test strip and/or permit the flow of oral fluid onto the lateral flow test strips when removed.
   a. Removal may be prompted by the device indicating a “full state”
   b. Removal may be prompted by the thermal control system with the reader after the device indicates full and/or once an adequate testing temperature has been achieved.
5. Controlling oral fluid flow rates in the lateral flow test strips.
   a. A precise nature of the laminated structure can apply very precise pressure to the reagent pads of the lateral flow test strip thus compressing the pad slightly and/or slowing and/or the flow rates into the lateral flow test strip providing more time for antibody antigen reactions to occur.
   b. A flow rates within lots of test strips can be somewhat variable due to variable densities of reagent pads. Compressing these pads to the same height can improve flow rate consistency.

General Process Description: This testing process of Non limiting optional embodiments can be summarized, as a non-limiting example, as optionally including one or more of:
1. Person performing the test (Tester) can removes a test device from the packaging, removes the cap and/or places the oral fluid collection in the test subject's mouth.
2. Tester opens the case on the electronic device and/or via a Graphical User Interface (GUI), initiates a test.
3. Tester waits until sufficient oral fluid has been collected (absorbed) by the test device as indicated by the color changing fill indicator and/or removes from mouth when complete and/or replaces the cap.
4. Tester removes the test initiation sticker and/or inserts into slot in reading device.
5. If not automatically initiated by insertion of test, Tester initiates test timing on GUI of electronic device immediately after insertion.
6. Electronic device can take an initial picture of the test device to determine specific test type information and/or lot numbers and/or any other specific information.
7. A accessory case can initiate internal lighting automatically when an image is being captured.
8. At the appropriate time a digital image or images can be taken of the test device.
9. Test and/or control line intensities can be electronically determined from the digital image.
10. A validity of the test can optionally be determined by analyzing control line intensity.
11. If valid, test line intensities can be electronically determined and/or subsequently positive or negative results determined for each drug tested for.
12. A Tester can be notified of the specific results.
13. If negative, next steps can optionally be determined by the organization in charge of the testing but may include collection of full test subject information as with positive shown below.
14. If positive, full test subject information can be collected by either or entering ID information into the device via the GUI or by taking digital images of the test subject, test subject's identification documents and/or test subjects vehicle if appropriate. Date, time and/or location determined by cellular GPS capabilities can also be added to the digital file.
15. Tester indicates via the GUI that testing is complete and/or the entire file is stored in the digital memory of the device in a secure format that is resistant to tampering. A file may then be transmitted to a centralized database via either cellular networks or Wi-Fi connections as an encrypted file for privacy and/or security proposes.
16. If necessary, the Tester can visually determine results as well.

In a non-limiting embodiment, the portable and/or wireless based lateral flow drug test strip system includes an optional test strip cartridge receptor, test format determination logic, test criteria determination logic, and digital camera hardware. A optional test strip cartridge receptor is responsive to a test strip cartridge having a test strip inserted therein. A test format determination logic determines an optical test format of the test strip. A test criteria determination logic determines an optical test criteria based upon the optical test format. A digital camera module is configured to capture an optical test image of the test strip and the calibration markings.

In accordance with another exemplary embodiment, a lateral test strip cartridge, that optionally includes a housing, is provided. A housing receives a selected test strip and is insertable into a digital camera reading position. A selected test strip has an associated optical testing format. A associated optical testing format of the selected test strip is different than an optical testing format associated with other test strips suitable for insertion into the housing. A optical testing format of the selected test strip has corresponding testing criteria for use by the digital camera hardware in connection with reading the test strip.

In accordance with another exemplary embodiment, a portable and/or wireless based lateral flow drug test strip system optionally includes an optional test strip cartridge receptor, test format determination logic, test criteria determination logic, and digital camera hardware module. A optional test strip cartridge receptor is responsive to a first type of test cartridge or test strip. A first type of test cartridge has a first test strip inserted therein. A second type of test cartridge is responsive to a second type of test cartridge having a second test strip inserted therein. A test format determination logic determines an optical test format of the first test strip or the second test strip, or additional test strips, each specific to one or more specified drugs, compounds, or metabolites thereof, to be tested. A test criteria determination logic determines an optical test criteria based upon the optical test format. A digital camera module is configured to capture an optical test image of either the first test strip or the second test strip, or additional pre specified test strips, each specific for one or more drugs, compounds or metabolites thereof. A digital camera module is separable from or integrated with a computer device including a display device (e.g. a smart phone, PDA, or digital camera).

In accordance with another exemplary embodiment, a method of using a portable and/or wireless based lateral flow drug test strip system having a digital camera to provide a digital captured image of a test strip and calibration/result markings within a test cartridge to a separable or integrated computer device having a display is provided. A method includes coupling an interface of the portable and/or wireless based lateral flow drug test strip system to an input port of the computer device, communicating data from the interface of the portable and/or wireless based lateral flow drug test strip system to the computer port of the computer device, and displaying an image on the display device. A displayed image is derived from the data received by the input port of the computer device from the interface of the portable and/or wireless based lateral flow drug test strip system. A portable and/or wireless based lateral flow drug test strip system includes an optional test strip cartridge receptor responsive to a first type of test cartridge having a first test strip inserted therein and responsive to a second type of test cartridge having a second test strip inserted therein.

TEST STRIP FEATURES AND COMPONENTS: A lateral flow test strip of Non limiting optional embodiments can be of any shape and dimensions, such as one or a combination of square, round, oval, polygonal, hexagonal, and the like, but preferably is a rectangular test strip.

A test strip of a test device of the non-limiting optional embodiments may comprise, at least in part, any bibulous or non-bibulous material, such as nylon, paper, glass fiber, dacron, polyester, nitrocellulose, polyethylene, olefin, or other thermoplastic materials such as polyvinyl chloride, polyvinyl acetate, copolymers of vinyl acetate and vinyl chloride, polyamide, polycarbonate, polystyrene, etc. In a preferred embodiment, at least one test strip material is nitrocellulose having a pore size of at least about 1 micron, more preferably of greater than about 5 microns, or about 8-12 microns. Very suitable nitrocellulose sheets having a nominal pore size of up to approximately 12 microns, are available commercially from, for example, Schleicher and Schuell GmbH.

A test strip used in context with the non-limiting optional embodiments can optionally include indicia that can include a designation for the test to be performed using the test strip. Such indicia may be printed on the test strip material using methods known in the art. Alternatively, indicia may be on other thin members, such as plastic or paper, that are attached to the test strip, such as by adhesives.

A test strip can include one or more materials. If a test strip comprises more than one material, the one or more materials are preferably in fluid communication. One material of a test strip may be overlaid on another material of the test strip, such as for example, filter paper overlaid on nitrocellulose. Alternatively or in addition, a test strip may include a region comprising one or more materials followed by a region comprising one or more different materials. In this case, the regions are in fluid communication and may or may not partially overlap one another.

A material or materials of the test strip can be bound to a support or solid surface such as found, for example, in thin-layer chromatography and may have an absorbent pad either as an integral part or in liquid contact. For example, a test strip may comprise nitrocellulose sheet “backed”, for example with a supporting sheet, such as a plastic sheet, to increase its handling strength. This can be manufactured by forming a thin layer of nitrocellulose on a sheet of backing material. A actual pore size of the nitrocellulose when backed in this manner will tend to be lower than that of the corresponding unbacked material. Alternatively, a pre-formed sheet of nitrocellulose and/or one or more other bibulous or non-bibulous materials can be attached to at least one supporting sheet, such as a sheet made of polymers (see, e.g., U.S. Pat. No. 5,656,503, entirely incorporated by reference). A supporting sheet can be transparent, translucent or opaque. In aspects of the non-limiting optional embodiments where the support sheet is transparent, the supporting sheet is preferably moisture impervious but can be moisture resistant or moisture pervious. In the non-limiting optional embodiments the test strip can be viewed through a window comprised of a transparent material such as glass, plastic, or mylar, but preferably break resistant.

In the following discussion strips of test strip material will be described by way of illustration and not limitation.

Generally, test strips of a test device of the non-limiting optional embodiments include a sample application zone and a test results determination region. A test results determination region can include either or both of one of more drug, compound, or metabolite detection zones and one or more control zones. Optionally, a test strip can include a reagent zone.

One or more specific binding members in the test results determination region of the test strip can be impregnated throughout the thickness of the bibulous or non-bibulous material in the test results determination region (for example, specific binding members for one or more drugs, compound, or metabolite can be impregnated throughout the thickness of the test strip material in one or more drug, compound, or metabolite detection zones, and specific binding members for one or more control drugs, compound, or metabolite can be impregnated throughout the thickness of the test strip material in one or more control zones, but that need not be the case). Such impregnation can enhance the extent to which the immobilized reagent can capture a drug, compound, or metabolite present in the migrating sample. Alternatively, reagents, including specific binding members and components of signal producing systems may be applied to the surface of the bibulous or non-bibulous material Impregnation of specific binding members into test strip materials or application of specific binding members onto test strip materials may be done manually or by machine.

Nitrocellulose has the advantage that a specific binding member in the test results determination zone can be immobilized without prior chemical treatment. If the porous solid phase material comprises paper, for example, the immobilization of the antibody in the test results determination zone can be performed by chemical coupling using, for example, CNBr, carbonyldiimidazole, or tresyl chloride.

Following the application of a specific binding member to the test results determination zone, the remainder of the porous solid phase material should be treated to block any remaining binding sites elsewhere. Blocking can be achieved by treatment with protein (for example bovine serum albumin or milk protein), or with polyvinylalcohol or ethanolamine, or any combination of these agents. A labeled reagent for the reagent zone can then be dispensed onto the dry carrier and will become mobile in the carrier when in the moist state. Between each of these various process steps (sensitization, application of unlabeled reagent, blocking and application of labeled reagent), the porous solid phase material should be dried.

To assist the free mobility of the labeled reagent when the test strip is moistened with the sample, the labeled reagent can be applied to the bibulous or non-bibulous material as a surface layer, rather than being impregnated in the thickness of the bibulous material. This can minimize interaction between the bibulous or non-bibulous material and the labeled reagent. For example, the bibulous or non-bibulous material can be pre-treated with a glazing material in the region to which the labeled reagent is to be applied. Glazing can be achieved, for example, by depositing an aqueous sugar or cellulose solution, for example of sucrose or lactose, on the carrier at the relevant portion, and drying (U.S. Pat. No. 5,656,503). A labeled reagent can then be applied to the glazed portion. A remainder of the carrier material should not be glazed.

A reagents can be applied to the carrier material in a variety of ways. Various “printing” techniques have previously been used or known in the art for application of liquid reagents to carriers, for example micro-syringes, pens using metered pumps, direct printing and ink-jet printing, and any of these techniques can be used in the present context. To facilitate manufacture, the carrier (for example sheet) can be treated with the reagents and then subdivided into one or more of smaller portions, layers, components, laminates, or other structures (for example small narrow strips each embodying the required reagent-containing zones) to provide a plurality of identical carrier units.

In embodiments where the drug, compound, or metabolite is detected by a signal producing system, such as by one or more enzymes that specifically react with the analyte, one or more components of the signal producing system can be bound to the drug, compound, or metabolite detection zone of the test strip material in the same manner as specific binding members are bound to the test strip material, as described above. Alternatively or in addition, components of the signal producing system that are included in the sample application zone, the reagent zone, or the drug, compound, or metabolite detection zone of the test strip, or that are included throughout the test strip, may be impregnated into one or more materials of the test strip. This can be achieved either by surface application of solutions of such components or by immersion of the one or more test strip materials into solutions of such components. Following one or more applications or one or more immersions, the test strip material is dried. Alternatively or in addition, components of the signal producing system that are included in the sample application zone, the reagent zone, or the drug, compound, or metabolite detection zone of the test strip, or that are included throughout the test strip, may be applied to the surface of one or more test strip materials of the test strip as was described for labeled reagents.

### Sample Application Zone

A sample application zone is an area of a test strip where a sample, such as a fluid sample, such as a biological fluid sample such as blood, serum, saliva, or urine, or a fluid derived from a biological sample, such as a throat or genital swab, is applied. A sample application zone can include a bibulous or non-bibulous material, such as filter paper, nitrocellulose, glass fibers, polyester or other appropriate materials. One or more materials of the sample application zone may perform a filtering function, such that large particles or cells are prevented from moving through the test strip. A sample application zone can be in direct or indirect fluid communication with the remainder of the test strip, including the test results determination zone. A direct or indirect fluid communication can be, for example, end-to-end communication, overlap communication, or overlap or end-to-end communication that involves another element, such as a fluid communication structure such as filter paper.

A sample application zone can also include compounds or molecules that may be necessary or desirable for optimal performance of the test, for example, buffers, stabilizers, surfactants, salts, reducing agents, or enzymes.

### Reagent Zone

A test strip can also include a reagent zone where reagents useful in the detection of a drug, compound, or metabolite can be provided immobilized (covalent or non-covalent immobilization) or not immobilized, particularly when in a fluid state. A reagent zone can be on a reagent pad, a separate segment of bibulous or non-bibulous material included on the test strip, or it can be a region of a bibulous or non-bibulous material of a test strip that also includes other zones, such as a drug, compound, or metabolite detection zone. In one aspect of non-limiting optional embodiments, the reagent zone can include a labeled specific binding member, such as antibodies or active fragments thereof attached or linked to a label. Such labeled specific binding members can be made using methods known in the art. A specific binding members can bind a drug, compound, or metabolite and/or can bind an optional compound.

In one example, the reagent zone can include two or more populations of colored beads. One population of colored beads is attached to an anti-rabbit IgG antibody or active fragment thereof and the other population of colored beads is attached to an anti-drug, compound, or metabolite antibody or active fragment thereof. A labeled anti-rabbit IgG antibody or antibody fragment is used for visual detection of a signal in the control zone of the test strip. A color signal in the control zone indicated that the sample has passed through the detection zone. A labeled anti-drug, compound, or metabolite antibody or fragment thereof provides a visual signal in the detection zone indicating the presence of drug, compound, or metabolite in the sample.

Other preferred embodiments are having anti-(drug of abuse) antibodies or active fragments thereof bound to a population of colored beads. More than one population of beads can be used as in the forgoing example to provide a visual signal in the detection zone and a second visual signal in the control zone. A two populations of beads can be the same or are different colors or be provided as a mixture of colors. Alternatively or in addition, different populations of beads bound to different antibodies or antibody fragments can be used to indicate the presence of more than one drug, compound, or metabolite in a sample by producing one or more visual signals in one or more detection zones.

In another aspect of non-limiting optional embodiments, the reagent zone includes the drug, compound, or metabolite or a drug, compound, or metabolite analog, bound to a population of colored beads. In this case, the drug, compound, or metabolite in the sample competes with the labeled drug, compound, or metabolite or drug, compound, or metabolite analog provided in the reagent zone for binding to a specific binding member in the test results determination zone. A reduced visual signal in comparison with an optional sample lacking drug, compound, or metabolite indicates the presence of drug, compound, or metabolite in the sample. More than one population of beads can be used as in the forgoing examples to provide a visual signal in the drug, compound, or metabolite detection zone and a second visual signal in the control zone. Alternatively or in addition, different populations of beads bound to different drugs, compound, or metabolite or drug, compound, or metabolite analogs can be used to indicate the presence of more than one drug, compound, or metabolite in a sample by producing one or more visual signals in one or more detection zones.

Preferred labels are beads such as metal particles, such as gold, or polymeric beads, such as colored beads, or particles of carbon black. Other labels include, for example, enzymes, chromophores or fluorophores such as they are known in the art, particularly in immunoassays, or later developed. A populations of beads are provided in powdered form on the reagent zone, which can include a bibulous material, such as filter paper, glass fibers, nylon, or nitrocellulose. These reagents are reversibly bound to the reagent zone because they can be mobilized when placed in contact with a fluid, such as a fluid sample passing along a test strip.

In another embodiment of non-limiting optional embodiments, the reagent zone can include components of a signal producing system, for example, catalysts, such as enzymes, cofactors, electron donors or acceptors, and/or indicator compounds.

A reagent zone can also include compounds or molecules that may be necessary or desirable for optimal performance of the test, for example, buffers (preferably dry buffers or conditioners), stabilizers, surfactants, salts, reducing agents, or enzymes.

### Test Results Determination Zone

A test results determination zone includes immobilized or not immobilized reagents that can detect the presence of the drug, compound, or metabolite being tested for, such as but not limited to, drugs of abuse (e.g., illegal, controlled, etc., drug or compound), metabolites, and antibodies. Such reagents are preferably in a dry state and can be covalently immobilized, non-covalently immobilized, or not immobilized in a fluid state. A test result determination zone can include either or both of one or more drug, compound, or metabolite detection zones and one or more control zones.

Depending on the particular format and drug, compound, or metabolite being tested for, a variety of reagents can be provided at the test results determination zone. For example, the test results determination zone can include specific binding members such as antibodies, enzymes, enzymatic substrates, coenzymes, enhancers, second enzymes, activators, cofactors, inhibitors, scavengers, metal ions, and the like. One or more of the reagents provided at the test results determination zone can be bound to the test strip material. Test strips including such reagents are known in the art and can be adapted to the test device of the present invention.

In a preferred aspect of the present invention, the one or more drug, compound, or metabolite detection zones of the test results determination zone include one or more immobilized (covalently or non-covalently immobilized) specific binding members that bind with one or more drugs, compound, or metabolite of interest, such as one or more drugs, hormones, antibodies, metabolites, or infectious agents, when the drugs, compound, or metabolite are also bound by specific binding members bound to a label as are provided in the reagent zone. Thus, in embodiments where the reagent zone contains one or more specific binding members for the analyte, the specific binding members of the reagent zone and drug, compound, or metabolite detection zone should bind with different epitopes on the drug, compound, or metabolite being tested for. For example, when a labeled specific binding member in the reagent zone binds with the drug, compound, or metabolite, then the immobilized specific binding member in the drug, compound, or metabolite detection zone should bind with another area of drug, compound, or metabolite. Thus, when drug, compound, or metabolite is present in the sample, the drug, compound, or metabolite will bind the labeled anti-drug, compound, or metabolite, which carried along to the test result determination zone at the drug, compound, or metabolite detection zone which binds with the immobilized anti-drug, compound, or metabolite to provide a visual readout.

A drug, compound, or metabolite detection zone can include substrates which change in an optical property (such as color, chemiluminescence or fluorescence) when a drug, compound, or metabolite is present. Such substrates are known in the art, such as, but not limited to, 1,2-phenylenediamine, 5-aminosalicylic acid, 3,3′,5,5′tetra methyl benzidine, or tolidine for peroxidase; 5-bromo-4-chloror-3-indolyl phosphate/nitroblue tetrazolium for alkaline phosphatase and 5-bromo-4-chloro-3-indolyl-beta-D-galactopyranoside, o-nitrophenyl-beta-D-galactopyranoside, napthol-AS-BI-beta-D-galactopyranoside, and 4-methyl-umbelliferyl-beta-D-galactopyranoside for beta galactosidase.

In embodiments where a drug, compound, or metabolite is detected by a signal producing system, one or more components of the signal producing system, such as enzymes, substrates, and/or indicators, can be provided in the drug, compound, or metabolite detection zone. Alternatively, the components of the signal producing system can be provided elsewhere in the test strip and can migrate to the drug, compound, or metabolite detection zone.

Optionally, the test results determination zone can include an optional control zone. A control zone can be upstream from, downstream from, or integral with the drug, compound, or metabolite detection zone of the test result determination zone. In the latter case, when drug, compound, or metabolite and control give a positive reaction, the control zone and/or drug, compound, or metabolite detection zone can form an indicia, such as a marking, indicator, or “+” sign for a positive reaction and a marking, indicator, “−” sign for a negative reaction based on the particular format of the assay, and the assay test strip or case or casing can also optionally include an indication or indication area that indicates that the one or more of the assays is not valid, either as the test or the control, optionally as a negative or positive control for one or more the assays as run on one or more of the test strips.

A control zone provides a result that indicates that the test on the test strip has performed correctly. In one preferred aspect of the present invention, the reagent zone includes a specific binding member that binds with a known drug, compound, or metabolite different from the drug, compound, or metabolite being tested for. For example, a rabbit-IgG may be provided in the reagent zone. A control zone can include immobilized (covalently or non-covalently) anti-rabbit-IgG antibody. In operation, when the labeled rabbit-IgG in the reagent zone is carried to the test result determination zone and the control zone therein, the labeled rabbit-IgG will bind with the immobilized an anti-rabbit-IgG and form a detectable signal.

A control zone can include substrates which change in an optical property (such as color, chemiluminescence or fluorescence) when an optional substance is present.

In one preferred aspect of the present invention, the test strip can include a results determination zone that includes an optional and a drug, compound, or metabolite detection zone, and a sample adulteration control zone. In another aspect of the present invention, a test strip can include a results determination zone that optionally includes an optional, and optionally an adulteration control zone. A second test strip can include an adulteration control zone and optionally an optional. Preferably, this second test strip includes both an adulteration control zone and an optional, but that need not be the case. In the instance where one or more first test strips can be used to detect a drug, compound, or metabolite other than an adulteration drug, compound, or metabolite and one or more second test strips can be used to detect an adulteration analyte, the test strips can be provided as multiple test strips or test strips that detect multiple drugs, compounds or metabolites.

### Orientation of Zones

A various zones of a test strip, including a sample application zone, one or more reagent zones, and one or more test result determination zones, including one or more drug, compound, or metabolite detection zones and optionally including one or more control and one or more adulteration zones, can be on a single strip of material, such as filter paper or nitrocellulose, or can be provided on separate pieces of material. A different zones can be made of the same or different material or a combination of materials, but preferably are selected from bibulous materials, such as filter paper, fiberglass mesh and nitrocellulose. A sample application zone preferably includes glass fibers, polyester or filter paper, the one or more reagent zones preferably include glass fibers, polyester or filter paper and the test results determination zone, including one or more drug, compound, or metabolite detection zones and optionally including one or more control, preferably include nitrocellulose.

Optionally, a fluid absorbing zone is included. A fluid absorbing zone preferably includes absorbent paper and is used to absorb fluid in a sample to drive fluid from the sample application zone through the reagent zone and the detection zone, which can optionally also include dry buffers or conditioning compositions.

Preferably, the zones are arranged as follows: sample application zone, one or more reagent zones, one or more test results determination zones, one or more control, one or more adulteration zones, and fluid absorbing zone. If the test results determination zone includes an optional, preferably it follows the drug, compound, or metabolite detection zone of the test result determination zone. All of these zones, or combinations thereof, can be provided in a single strip of a single material. Alternatively, the zones are made of different materials and are linked together in fluid communication. For example, the different zones can be in direct or indirect fluid communication. In this instance, the different zones can be jointed end-to-end to be in fluid communication, overlapped to be in fluid communication, or be communicated by another member, such an joining material, which is preferably bibulous such as filter paper, fiberglass or nitrocellulose. In using a joining material, a joining material may communicate fluid from end-to-end joined zones or materials including such zones, end-to-end joined zones or materials including such zones that are not in fluid communication, or join zones or materials that include such zones that are overlapped (such as but not limited to from top to bottom) but not in fluid communication.

When and if a test strip includes an adulteration control zone, the adulteration control zone can be placed before or after the results determination zone. When an optional is present in the results determination zone on such a test strip, then the adulteration control zone is preferably before the control zone, but that need not be the case. In non-limiting optional embodiments where a test strip is an optional test strip for the determination of an adulteration drug, compound, or metabolite and/or an optional, then the adulteration control zone can be placed before or after the control zone, but is preferably before the control zone.

### Methods of Detecting of a Drug, Compound, or Metabolite in a Sample

A device of non-limiting optional embodiments can be used to collect a sample, transfer the sample to a test strip sample receiving zone and optionally mix the sample with one or more reagents, such as dry buffer or conditioner. A sample or sample and one or more reagents can then be conducted to a test element within a test strip to detect one or more drugs, compounds, or metabolites in the sample, preferably a sample application zone of a test strip. A sample can be liquid or colloidal. Examples of liquid or fluid samples that can be applied to the test strip can include blood, serum, saliva, or urine.

To collect a sample a fluid or colloidal sample can be applied via various techniques, for example pipeting, pouring or by use of a dropper. Alternatively a sample collection device can be used to collect a sample and transfer the sample onto the test strip. A sample collection device can be of different structures but is preferably a swab. A swab can be used to collect the sample onto the swab head by different embodiments such as for example dipping, swiping or swabbing. A swab with sample can be applied to the test strip that can optionally contain one or more reagents, or with dry buffer added to the sample.

### DESCRIPTION OF THE DRAWINGS

In a particular embodiment, the disclosure is directed to an automated, portable, and wireless lateral flow test strip drug testing platform for optical analysis of pre calibrated lateral flow test strips for illegal or controlled substance drug, compound, and/or metabolite threshold testing assays utilizing a smart phone provided with or in a protective case with digital image recognition software algorithms for qualitative and/or quantitative data test analysis and result reporting in a customizable software suite, with integrated alternative test strip/casing positioning for calibrated/result markings for digital image analysis to provide drug testing results in real time using dry buffer or conditioning of saliva or body fluid samples for the drug test strips for one or more specified drugs, compounds, or metabolites thereof.

Non limiting optional embodiments optionally provides a portable drug testing platform for digital image capture and analysis of pre-calibrated/quantitation of lateral flow drug test strips using dry buffer or pre-conditioning of saliva or body fluid test samples. A platform can optionally include digital camera hardware with digital components that record the pre-calibrated/quantitative test strips optionally including active chemistry and specific for one or more drugs, compounds, or metabolites; dry buffer or conditioning media for preparing the test sample; software for interfacing with the user, and an image processing and computing device to interface with the digital camera.

In the particular embodiment, the system accepts a broad range of lateral flow test strips. A test sample (e.g., saliva or body fluid) is taken from the person being tested and added to one or more of the test cartridge or the test strip, that is provided with dry buffer or conditioners to set up the sample for addition to the test strip for testing. A lateral test strip or test cartridge is provided with the conditioned test sample at a designated area and the sample then continues by timing or indicator to the designated or proper position for interaction with the drug analysis components of the test strip to react the sample to provide the indication of a positive, negative, and/or threshold amount of the drug, compound, or metabolite being tested by a particular test strip. A test strip or casing of the test strip is provided with calibration, sufficient test sample, and result indicator markings to show the result of the test for each test strip and corresponding drug, compound or metabolite being tested.

A system then continues the drug testing by taking a digital image of the sample conditioned and run on each test strip with the digital camera positioned, optionally, via the tester, case and/or housing. Illumination can optionally be provided by the digital camera or a separate illumination source. Digital image data of the test strip result, additional identifying information including one or more of identification of the person being tested, information about the tester, the location, the drug testing being done, and the like, and this image and other data is collected and stored in the digital camera, data memory storage, and/or a cloud based or separate data memory storage device. A digital image data is then processed using a host device (e.g. dedicated smart phone, PDA, laptop, cellular phone, or the like) using processing capabilities in conjunction with the software component of the system. Software pre-loaded onto the smart phone or processor provides the processing instructions and compares image analysis data to pre-defined calibration data, yielding a qualitative or quantitative result, e.g., but not limited to positive, negative, over or below one or more threshold concentrations or amounts, and the like. A system can interface with the host device through several different physical standards. These standards include industry standards such as Personal Computer Memory Card International Association (PCMCIA), Universal Serial Bus (USB), Serial, Secure Digital, BlueTooth™, one or a combination of optical, magnetic, or solid state data drives, Wi-Fi or other company specific standards such as the Handspring Springboard Platform™.

In another embodiment, software is automated for later flow test strip digital imaging for cross-field testing compatibility. This system can provide compatibility with a wide array of commercial or custom lateral flow strips. A system digitizes and objectively quantifies results from tests (such as test strips that can optionally be conventionally read by a human manually); stores original and modified digital image and data into memory for review; and enhances test processing by executing image processing algorithms.

A exemplary embodiment provides a fully self-contained, portable, wireless, and adaptable handheld automated drug testing system, apparatus, and method for setting up (optionally via display panel and/or GUI), obtaining drug test sample (e.g., saliva or body fluid) from person being tested, adding drug test sample, conditioning drug test sample, adding conditioned drug test sample to test strip, confirming sufficient conditioned sample added to test strip, running conditioned sample on test strip to show visual result of test, acquiring digital image of test strip indicating test strip result as well as additional image or other data providing information about testing, tester, location, etc., analyzing the digital image and/or data, interpreting the digital image and/or data, and reporting drug test results gathered from drug testing strips via GUI, wireless data transmission, memory storage, or other electronic communication.

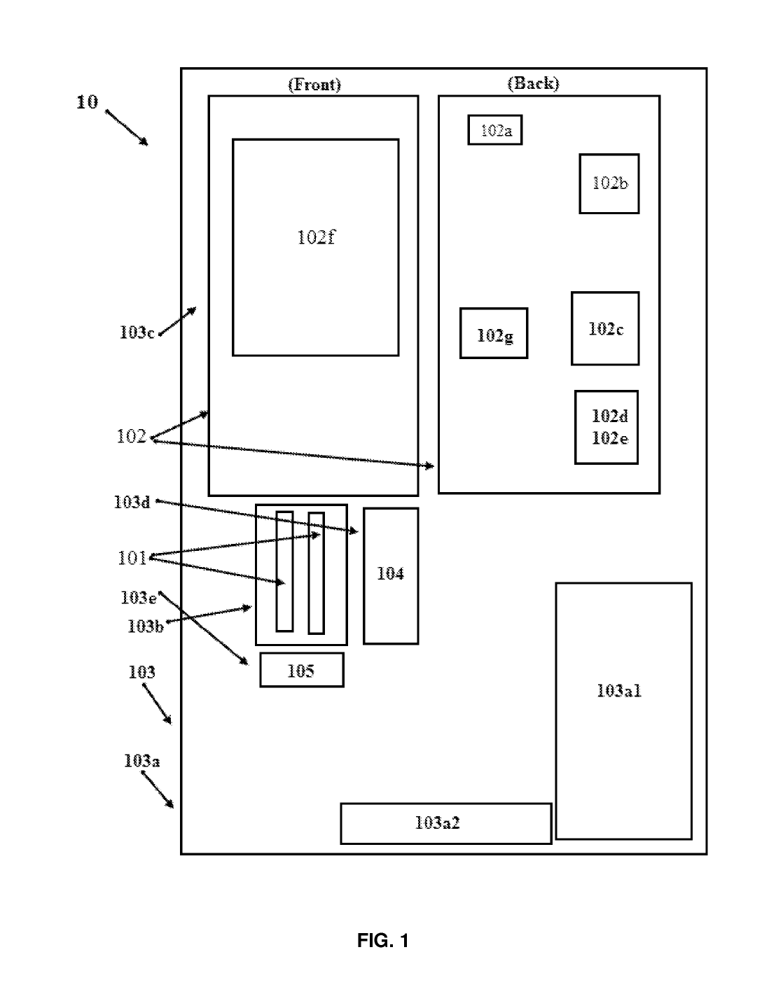

FIG. 1 depicts a non-limiting embodiment of Non limiting optional embodiments as a portable lateral flow drug testing system 10 schematic comprising drug test strips 101; smart phone 102 comprising digital camera hardware 102a; computer processor 102b, data memory storage 102c, communication hardware 102d; software 102e; display/GUI 102f; and/or GPS 102g; case/housing 103 comprising outer shell 103a with optional instructions label 103a1; and/or optional identification tag or 2D or 3D code and/or label 103a2; test cartridge/test strip receptors 103b, smart phone dock 103c, optional illumination dock or positioning component 103d; and/or optional heater dock 103e: optional illumination hardware 104; and optional heater hardware 105. A exemplary embodiment allows the user or tester to hold the system case in one hand, insert the test strip/cartridge into test strip/cartridge receptor using their other hand and activate the automated software routine.

A system 10 can alternatively interface with electronics devices other than a smart phone, such as but not limited to, tablets, laptop and desktop computers, cellular phones, or other devices that have data input and display components. A communications interface with these other devices can be through standard ports such as PCMCIA, USB, Firewire, infrared, Wi-Fi, Bluetooth, or other custom communication protocols. A communications hardware can include electronics such as a Field Programmable Gate Array (FPGA) and/or microcontroller that can be re-configured to utilize the appropriate communication protocol. Multiple protocols can be provided in the communications or processor hardware and can be accessible through standard pre-installed electrical connectors.

In addition to the adaptability of the cartridge to different test strip formats, the case/housing's internal track design for the cartridge receptors, which accepts and guides the cartridges or test strips, can itself be capable of accepting a variety of cartridges or test strips. A internal track design can be a removable injection molded or machined piece that can be replaced dependent upon the user's preferences. A internal track design in conjunction with the cartridge can serve to accurately position each cartridge or test strip according to pre-defined positioning criteria. A track design provides three-axis positioning accuracy of each inserted adapter using a floating tensioning mechanism.

An exemplary guide has various channels with different depths and widths. A guide positions various cartridges or test strips to a centered or properly oriented position for imaging.

In one exemplary embodiment, the insertion method allows the user to view the strip during insertion and ensures proper orientation. This method also helps reduce the potential for ambient light leakage into the interior of the housing from external light sources. A insertion method can also allow for longer cartridges to be accommodated as the back wall can be removed to allow the cartridge to pass completely through the case/housing. Insertion of the cartridge or test strip by the user is aided by an internal track design that guides the universal adapter along a predefined course. Insertion of the cartridge fully into the appropriate track can activate the reader via a micro switch. This can initiate the software routine, which then images the cartridge or test strip, retrieves the signature label data, references the stored databases, and completes the optical imaging and processing of the sample.

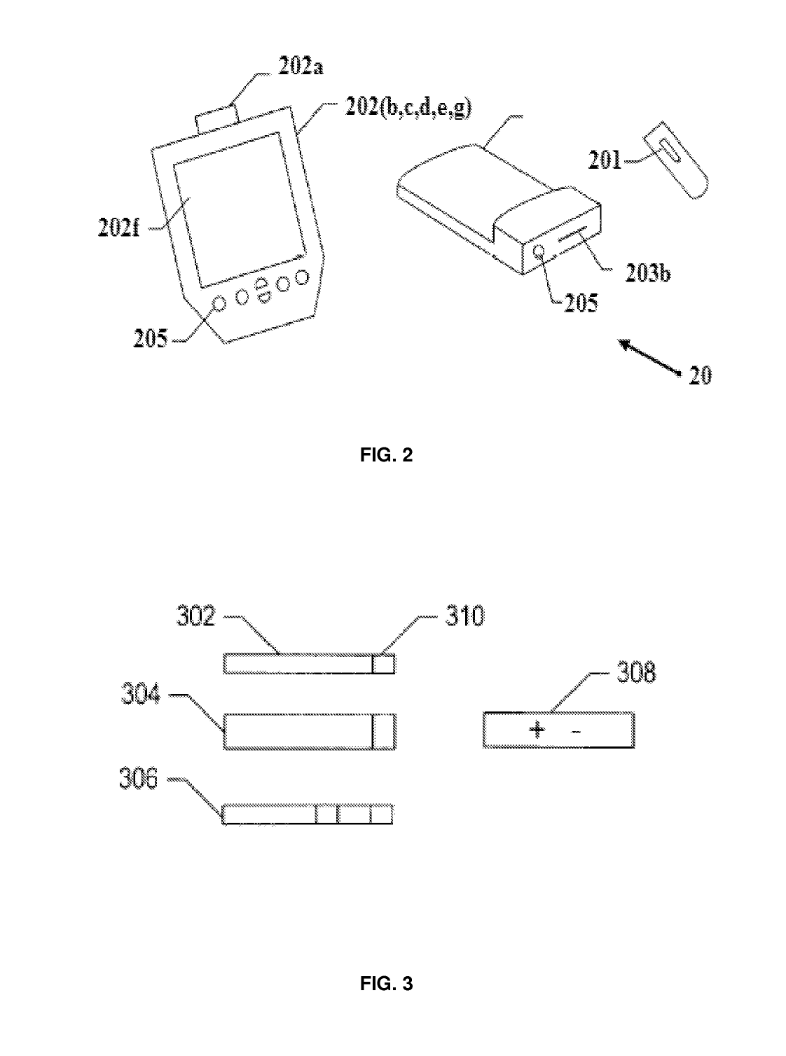

FIG. 2 depicts another optional exemplary embodiment of a lateral flow drug test system 20 of non-limiting optional embodiments. A test strip/cartridge 201 can be inserted into test strip/cartridge receptor 203b which is oriented for calibrated image capture by digital camera hardware 202a. A digital camera hardware 202a is provided in smart phone 202 which also optionally includes one or more of computer processor 202b, data memory storage 202c, communication hardware 202d; software 202e; display/GUI 202f; and/or GPS 102g. A smart phone 202 is programmed to collect, store, analyze, and report test data comprising set up, calibration, sample preparation and analysis, data collection, and test results using digital images taken with the digital camera hardware 202a, where one or more of test parameters, test software, and testing data can be manipulated using the display/GUI 202f touch screen or button interface.

Indicator lights 205 can be used to indicate proper or improper insertion of a test strip/cartridge 201 into a test strip/cartridge receptor 203b, proper or improper sample application or running on the test strip; or other states associated with the reader functionality.

A cartridge 201 can accept lateral flow test strips of various types. Alternately, test cartridges and/or test strips of various types, sizes, shapes, and chemistries for one or more drug tests can be provided for reading by the portable and/or wireless based lateral flow drug test strip system. A cartridge can be used to hold a several types of test strips without the use of a separate adapter. This component can be injection molded and can be capable of having a variety of test strips inserted into the clamshell design. An optional adapter provided with the cartridge can position the strips in such a fashion as to position the test and control lines or zones of interest in view of the proper imaging window. Alternately, multiple windows can be provided. A top portion of the adapter can overlay a bottom portion, enclosing the test strip. A bottom portion can include a channel for guiding the test sample.

Additionally, each test strip, cartridge or adapter can have a signature label region that can include a discrete consistent region for coding. This region can be a variety of sizes dependent upon the amount of data storage needed. A coding can include a series of raised, flat, or depressed bar, 2D, or 3D code, or color dots in a one or a variety of colors. These dots can take an irregular or a regular shape and serve to uniquely identify the test strip, cartridge, or adaptor. In other exemplary embodiments, the coding can be an image, lettering, or other identifying marking. Each signature label can be unique and upon manufacture can be imaged and referenced to the appropriate database. These unique signatures can be captured during the imaging process and can then be compared to the pre-established database for identification purposes. Data stored in these signature labels or in the associated database can include predetermined test lot data, calibration coefficients, optical standards, expiration dates, illumination requirements, etc. Additionally, this signature label region can include temperature or chemical sensitive dyes that alter their color dependent upon exposure history and can serve as an automated quality control measure.

FIG. 3 depicts various types of lateral flow strips such as strips 302 and 306, the strips can have different sizes as indicated by strips 304 and 306. In some exemplary embodiments, the active area of the test strip 310 can include an adjacent casing 308 where the casing or test strip or casing can be provided with markings or color coding or changes that indicate one or more of sufficient sample volume or run time, positive or negative result, or one or more threshold drug test concentrations met or not met. In some cases, the marking or color to which it changes can indicate drug tested concentration. In other exemplary cases, the location of the marking or color change can be important. In still a further example, a shape or lack of a shape of the sample flow positioning can indicate the result of a test.

A imaging system can capture an image of the test strip and optional casing and transfer that image to analysis systems provided in the computer processor and software. A image system can include a normal visual spectra, infrared, ultraviolet, CCD, CMOS, or other imaging circuitry.

A system or case/housing can also have a gate to block light from outside the case/housing. Internal light sources such as halogen, fluorescent, or white light, or LEDs can be provided in various wavelengths to illuminate a test strip and provide enhanced frequency profiles for observing various test strips.

In general, the illumination source can direct light or electromagnetic energy on a test strip or cartridge inserted into test strip/cartridge receptor in the case or housing. A illumination source can vary the intensity or wavelength of light in accordance with the testing criteria associated with the test strip or cartridge. In other embodiments, a white light source or set of sources covering multiple wavelengths can be used for each test strip or cartridge, which can be provided by the digital camera or other light source.

Light reflecting from the test strip, casing and/or cartridge can be collected by the digital camera and converted to a digital image data files or files by the processor and stored on the memory data storage. A data can be processed by the processor and/or transferred to a computer for further analysis.

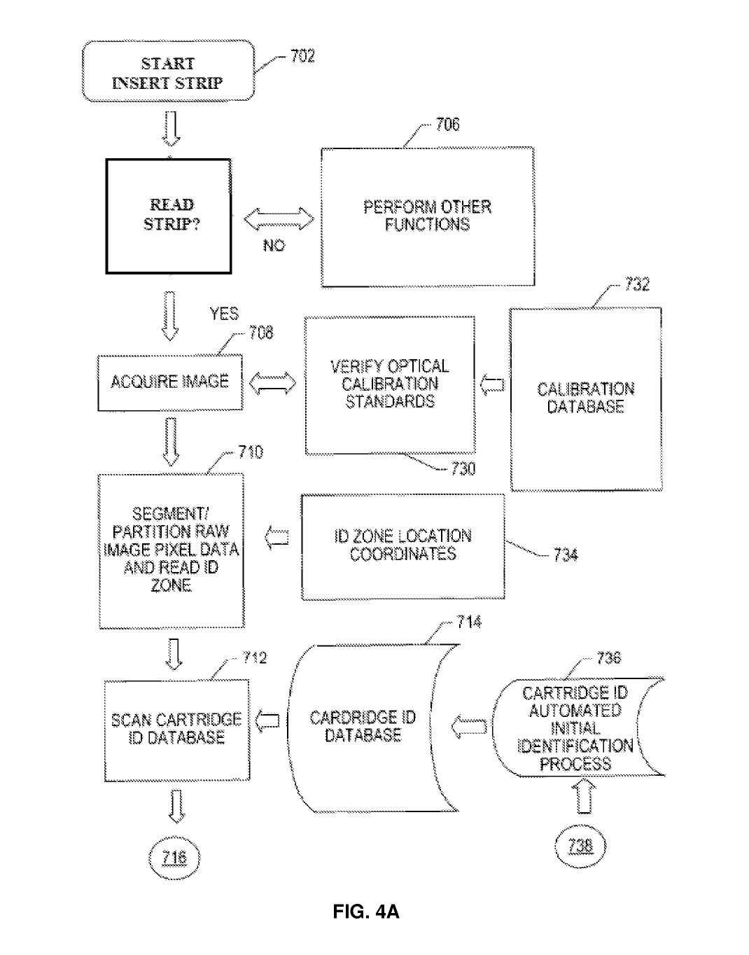

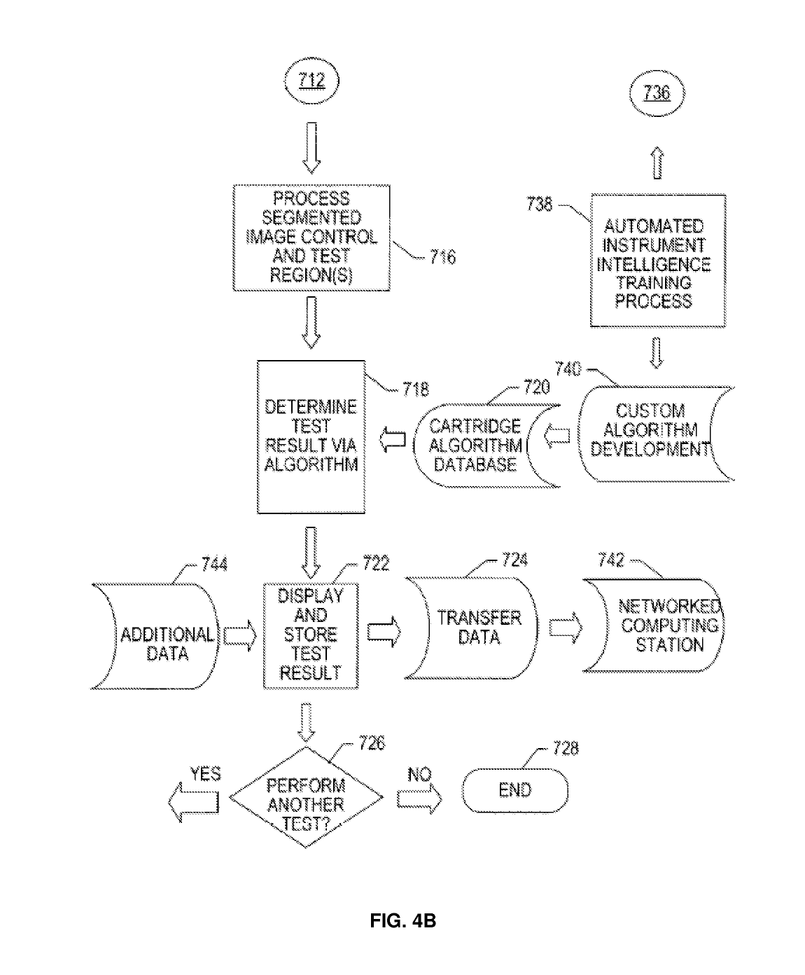

FIGS. 4A and 4B depict a flow diagram for an exemplary method or system of Non limiting optional embodiments to read and analyze a test strip. A method begins as shown at step 702. A process can start through initiation by a user or tester, opening the case or housing, obtaining and conditioning of a sample from a person to be drug tested, turning on the smart phone, selecting or inserting the conditioned sample added test strip to a cartridge or a test strip receptor in the housing, properly oriented and/or according to instructions provided with the system, and then confirming that the test is set up and ready for reading by recording a digital image of the prepared test strip using the digital camera hardware of the system, e.g., upon the expiration of a time period, indication by the GUI or the test strip or casing, or some other event. A determination is thus made as to whether to read a test strip, casing and/or cartridge, as shown at step 704. A user or tester can be prompted or a switch indicating the presence of a test strip or cartridge can be checked. If the cartridge is not to be read, other options can be provided to the user as shown at step 706, such as providing one or more of identification of tester or tested, location of test, type of test, types of test strips, codes or identification of test strips or cartridges, calibrating or setting up test, taking, recording, and/or preparing the sample, and the like.

When the test strip or cartridge is ready to be read, the digital camera hardware can acquire an image as shown at step 708. In conjunction with the acquisition of the image, the system can verify calibrations as shown at step 730. This verification can involve reviewing the image in the region of calibration areas on the instrument or cartridge. A calibration can further use a calibration database 732 to store and retrieve calibration data, such as known standards results or optical qualities.

A image can be segmented and an optional identity zone can be isolated or read as shown at step 710. A identity zone can be a pre-defined location or set of coordinates 734, or can be determined by the software automatically. This identity zone can contain a bar code, a set of colored shapes, images, or lettering. Utilizing the image data from the identity zone, the identity of the test strip and/or cartridge can be determined, as shown at step 712. For example, result markings or colors in the identity zone can be matched with patterns stored in a database 714. A database 714 can store information about the test strip or cartridge, such as test strip or cartridge type, lot, expiration date, and imaging protocol. In addition, the identity database 714 can be adapted through an initial identification process as shown in step 736. For new test strips or cartridges or to adapt existing data, an automated instrument intelligence training process, as shown in step 738, can be performed. This process can act to adapt the database 714.

Using the identification data, the system can process the image as shown at step 716. A test determination logic can be used to determine an optical test format. A format may, for example, direct the processing of specific zones in the image, such as an optional region and a sample test result region. A processing can result in a test result as shown in step 718. A test criteria determination logic can be used to determine an optical test criteria. This test criteria can be determined through reference to a cartridge algorithm database 720. A cartridge database 720 may, for example, hold calibration sequences, processing algorithms, marking or color mappings, patterns for pattern recognition, or other suitable test criteria for the received test strip or cartridge. Evaluation logic can apply the test criteria to data acquired from the image. A processing and determining steps can be performed on the reader or in coupled computation circuitry such as within a host smart phone. Users or testers can also adapt the cartridge algorithm database 720 using the automated instrument intelligence training process 738 and a custom algorithm development step 740.

A test results are then displayed as shown at step 722. Additional data 744 can be associated with the results such as Global Positioning System (GPS) data, pH, temperatures, humidity, and other environmental conditions. A reader can include additional instruments with sensors appropriate for gathering the additional data or the data can be acquired from other sources. These results can be displayed on the smart phone or by other computing circuitry. A display image can be derived from the data received by the input port. A results can be further uploaded to a server, personal computer, or other computational circuitry 742 for long term storage and access, as shown at step 724. Access can be provided through a website or other network connection.

In one embodiment, the data is stored on the smart phone until it is transferred to either the host device or is uploaded to a secure site. This upload process can include communication via wireless technologies such as Wi-Fi, 802.11, or other similar standards.

Data can be transferred in its raw format for secondary analysis along with the pre-processed data. A data can include, in addition to the test results and image, information identifying the user, time of test, location, error status, and/or environmental conditions at time of test.

A system can prompt the user for performing another test, as shown at step 726. If another test is to be performed, the system can return to step 702. If another test is not performed, the method can end as shown at step 728.

Software can serve to automate both the user interface experience and the process of data acquisition and reporting. A software can be built upon a premise that the user simply inserts the cartridge into the reader and the remainder of the tasks are either automated or prompted via the user interface. A process can be performed on the reader, on the portable or multipurpose circuitry, or on a combination of the reader and the circuitry.

Image processing algorithms can be used to interpret the image data against predetermined criteria. The image processing algorithms that can be used are numerous and vary depending upon the type of test being run, the sample conditions, the desired result sensitivity and other variable affects such as background contamination, signal to noise conditions, and overall quality of the test sample. Examples of the basic algorithms include the following:

1) Signal Averaging over entire Area—Average pixel intensities for each optical channel (e.g. Red, Green, Blue) are recorded for the entire selected areas. These are reported as a ratio to one another (i.e. R/G or R/B or R+G/B etc.) or simply as R,G,B values.

2) High/Low difference averaging—A lowest pixel value is subtracted from the highest pixel value for each pixel row and/or column and the difference is reported and averaged across either all the rows or all the columns or any combination therein.

3) Variance Measurement of Averages—A statistical variance in the pixel intensity for each row or column of pixel data is calculated and the variances are averaged either across rows, columns, or combinations therein. Additional weight or scaling factors can be given to a particular channel (RGB) versus another.

4) Partitioning and Averaging—The average pixel intensity is determined for a region assumed to contain both a high region and a low region, although the location of these high and low regions may not be known. The average pixel intensity is then used as a threshold to partition pixels in the high region from those in the low region. The pixels in each partitioned region are then averaged to find the average intensity of the low and the high regions. Optionally, this process may be repeated within each region to eliminate borderline pixels, and therefore calculate intensity averages more representative of each region.

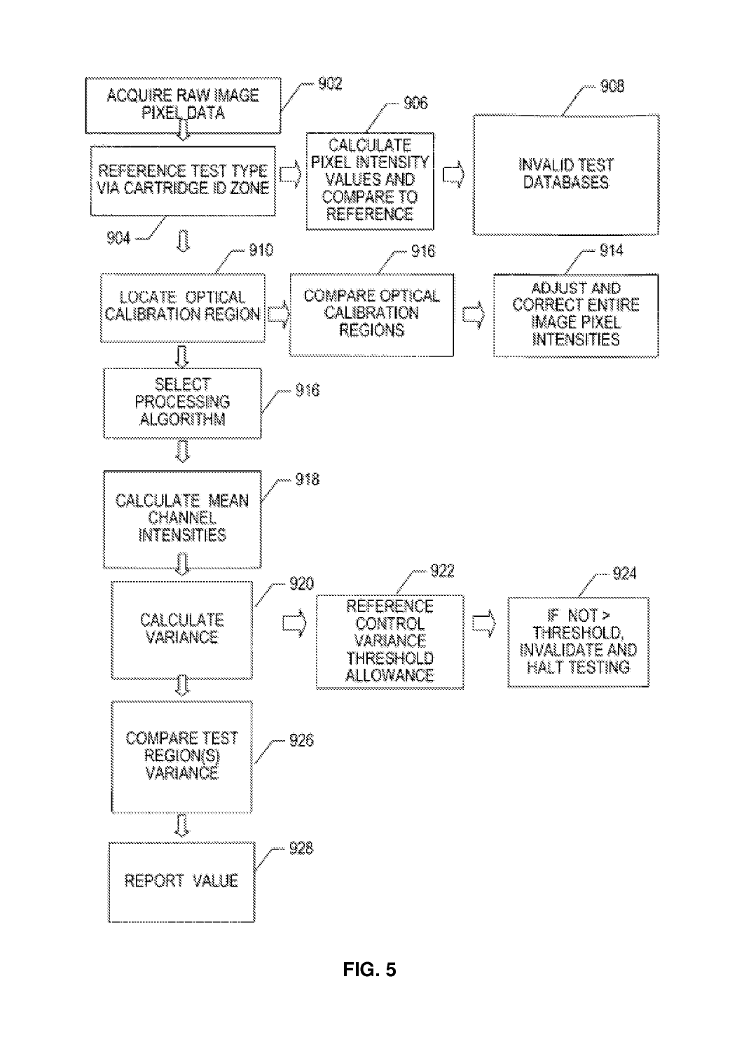

FIG. 5 depicts an exemplary image processing method. An image is acquired, as shown at step 902. A image detector can be the digital camera or other detector. A test strip or cartridge type is determined via the identity zone of the image, as shown at step 904. A test can also be performed on the test strip or cartridge to determine if the cartridge or test is valid. For example, as shown at step 906, mean segment region pixel intensity can be determined and compared to references within an invalid test database 908. A invalid test database 908 can store high and low mean pixel thresholds, cutoffs, and ratios for individual tests.

Once the test strip or cartridge is identified, an optical calibration region can be determined, as shown at step 910. Pixel intensity values can be determined for the optical calibration regions and compared to a calibration database, as shown in step 912. Adjustments can be made to correct the image, such as adjusting ratios and intensities of the image so that the calibration regions match pre-set reference levels.

Based on the test strip or cartridge identity, a processing algorithm can be selected from a test database, as shown at step 916. In one exemplary algorithm, the pixel row and column mean channel intensities can be calculated for individual regions, as shown at step 918. As shown at step 920, the variance in row and column pixel intensities can be predetermined and/or calculated for individual regions. These variances can be compared to threshold allowance values, as shown in step 922. If the control variance is not greater or lower than a predetermined and/or calculated threshold, the test can be invalidated and halted.

A test region variance can be compared to a set of test result threshold values, as shown at step 926. These thresholds can be predetermined thresholds for a particular cartridge type. After performing the comparison to the threshold level a result is determined. A test results and other information can then be reported to a user, as shown at step 928.

A system or method is designed to accommodate multiple unique test strips, cartridges and/or configurations and form factors. This allows test strips or cartridges designed with different physical parameters to be used with the system and hence be inserted and processed. A system, in addition to illuminating the test strip or cartridge and taking digital images, can also incorporate analog and digital inputs and outputs for plug-ins, such as environmental and physiologic sensors (e.g. thermometers, gas detectors, EKG data, GPS modules, RFID readers).

A system software can perform one or more of several distinct functions. These functions include one or more of the following, but are not limited to: 1) controlling the illumination and detection algorithms; 2) providing or creating a customizable user interface; 3) and providing image and/or pattern recognition and/or color mapping for conversion of optical images into quantitative graphical and numerical data. A software can be any known and suitable operating system (e.g., Windows™, Android™, iOs™ (e.g., iPhone, iPad, iPod); Linux™, and the like), however, the principles behind the algorithms are programming language agnostic. A initial routine for the software is to take a one or more images of the cartridge automatically when set up or when prompted through the user interface. A pre-defined region of this image can be correlated or dedicated to the code, ID, or signature label or marking that identifies the test strip or cartridge type. This can be done using visual, pattern, image, or spectral analysis of discrete sections of the code region that in turn provides a sequence of pixel or other image intensity or pattern values. This portion of the code or ID sequence is compared to a database of codes to determine the remaining processing parameters and test criteria for the cartridge. In particular, this code identifies the active test, ID, and/or control markings, regions, or coding of the test strip, casing, or cartridge, which can define one or more of testing, calibration, result, ID, drug, and/or illumination settings or markings, time resolved analysis procedures, and/or stores lot and expiration data.

A software can also be capable of being set up, preset, or trained via a calibration and/or setup routine in which known positives and negatives are provided, determined, run and a processing and calibrating algorithm is provided to differentiate and quantify the degree to which a sample is positive or negative. This system is capable of, with one or more images, for identifying the test strip or cartridge type, automatically retrieving the appropriate database of analysis parameters, and reading the test and/or control regions of the test strip or cartridge.

### Example 1

### Test Strip Features and Components

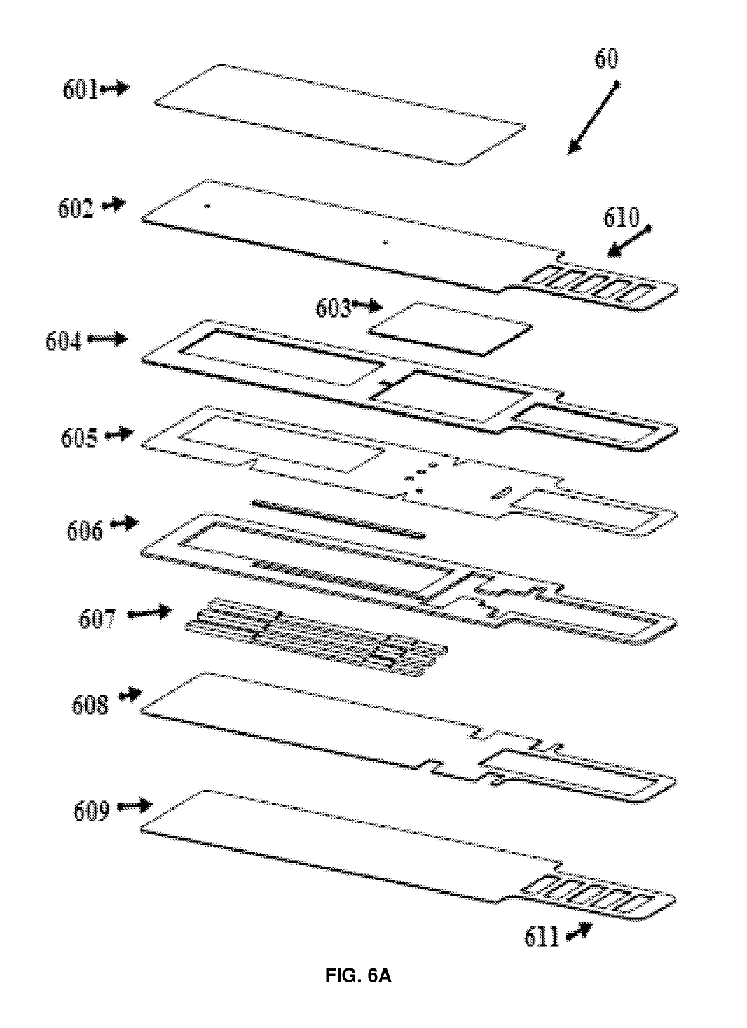

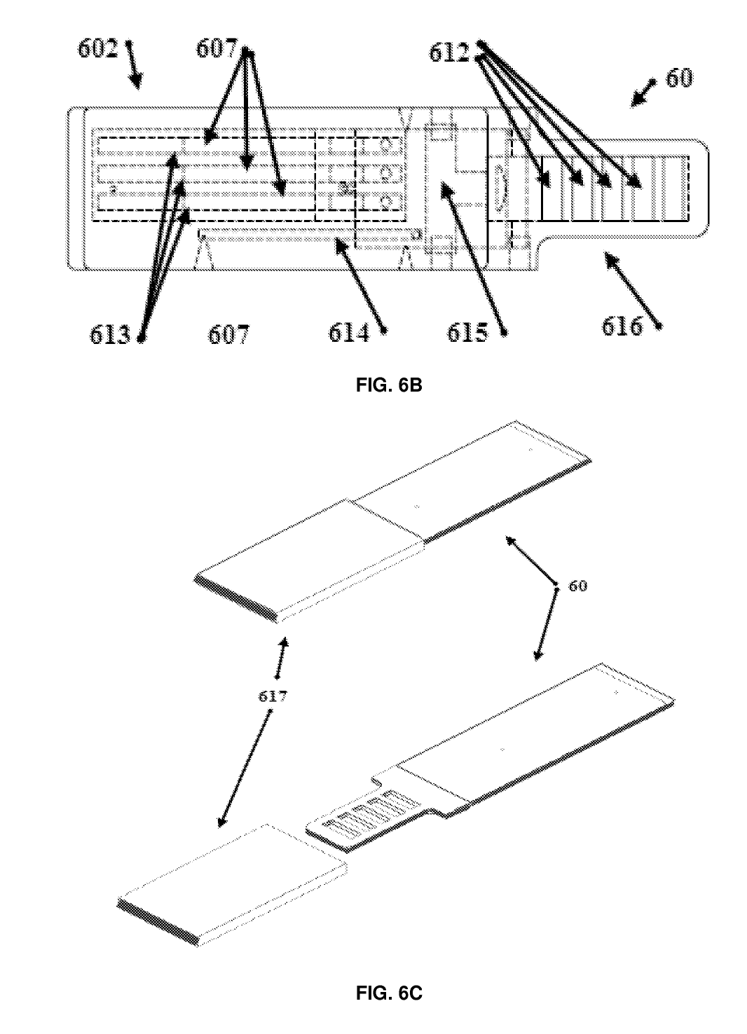

A non-limiting example of a lateral flow test strip of non limiting optional embodiments is provided with reference to FIGS. 6A-6C.

At least one test strip material can optionally comprise nitrocellulose or other suitable material(s) having a pore size of at least about 1 micron or other suitable size depending on the application or use.

As shown in exploded view FIG. 6A and top view of assembled test strip FIG. 6B, a test strip 60 used in context with non-limiting optional embodiments can optionally include a transparent cover layer or casing component 601, a transparent top layer/casing 602 with sample application windows 610, sample preparation/dry buffer/conditioner strip 603, first compartment template casing 604, second compartment template casing 605, third compartment template casing 606, with spacers 615 between 605 and 606, assay path channels 607, first bottom casing template 608, and second bottom casing template 608 with bottom sample application compartments 611. As shown in FIGS. 6A and 6B providing exploded and top views of assembled test strip/casing 60, sample application wells 612 are provided by the combination of sample application windows 610, first compartment template casing 604, second compartment template casing 605, third compartment template casing 606, first bottom casing template 608, and second bottom casing template 609 with bottom sample application compartments 611.

As shown in FIGS. 6A and 6B providing exploded and top views of assembled test strip/casing 60, sample buffer/conditioning manifold 615 is provided by the combination of transparent top layer/casing 602, sample preparation/dry buffer/conditioner strip 603 for conditioning sample, first compartment template casing 604, second compartment template casing 605 with holes for entering manifold 605, third compartment template casing 606 comprising manifold area 615 in fluid communication with assay path channels 607, first bottom casing template 608 supporting manifold 615 in fluid communication with assay channels 607.

As shown in FIGS. 6A and 6B providing exploded and top views of assembled test strip/casing 60, assay channels 607 are provided by the combination of transparent top layer/casing 602, the windows in first compartment template casing 604, second compartment template casing 605, and third compartment template casing 606; assay path channels 607, and first bottom casing template 608.

As shown in FIGS. 6A and 6B providing exploded and top views of assembled test strip/casing 60, test result indicator positions 613 are provided by the combination of transparent top layer/casing 602, windows provided in first compartment template casing 604, second compartment template casing 605, and third compartment template casing 606; assay path channels 607, and first bottom casing template 608.

As shown in FIG. 6C a removable end protective cover 617 can be provided to do one or more of protect, seal, keep sterilized, prevent moisture or other contamination and the like.

Indicia that can include a designation for the test to be performed using the test strip 60. Such indicia may be printed on the test strip material using methods known in the art. Alternatively, indicia may be on other thin members, such as plastic or paper, that are attached to the test strip, such as by adhesives.

A test strip can include one or more materials, as presented above and shown in FIGS. 6A and 6B. If a test strip comprises more than one material, the one or more materials are preferably in fluid communication for movement of the sample through the test strip. One material of a test strip may be overlaid on another material of the test strip, such as for example, filter paper overlaid on nitrocellulose. Alternatively or in addition, a test strip may include a region comprising one or more materials followed by a region comprising one or more different materials. In this case, the regions are in fluid communication and may or may not partially overlap one another.

A material or materials of the test strip can be bound to a support or solid surface such as shown in FIGS. 6A-6B, for example, and may have an absorbent pad either as an integral part or in liquid contact. For example, as shown in FIGS. 6A and 6B, a test strip may comprise nitrocellulose sheet “backed”, for example with a supporting sheet, such as a plastic sheet, to increase its handling strength. This can be manufactured by forming a thin layer of nitrocellulose on a sheet of backing material or by a pressing machine or device, such as a high speed press. A actual pore size of the nitrocellulose when backed in this manner will tend to be lower than that of the corresponding unbacked material. Alternatively, a pre-formed sheet of nitrocellulose and/or one or more other bibulous or non-bibulous materials can be attached to at least one supporting sheet, such as a sheet made of polymers (see, e.g., U.S. Pat. No. 5,656,503, entirely incorporated by reference). A cover, intermediate and/or supporting sheet can be transparent, translucent or opaque, and/or provided with windows, gaps or spaces. In the aspect of non-limiting optional embodiments where the support sheet is transparent, the supporting sheet is preferably moisture impervious but can be moisture resistant or moisture pervious. In another embodiment of non-limiting optional embodiments the test strip can be viewed through a window comprised of a transparent material such as glass, plastic, or mylar, but preferably break resistant.

In the following discussion strips of test strip material will be described by way of illustration and not limitation, e.g., as shown in FIGS. 6A and 6B.

Generally, test strips of a test device of non-limiting optional embodiments include one or more sample application zones (610, 611, and/or 612) and one or more test results determination regions (613 or other part of 607). Test results determination regions can include either or both of one of more drug, compound, or metabolite detection zones (e.g., 613), that can exclude or include additional or integrated optional control zones. Optionally, a test strip can include one or more reagent zones, e.g., a buffer, (e.g., a dry buffer) or conditioning compounds zone (e.g., 603 and/or 615).

One or more specific binding members in the test results determination regions of the test strip can be impregnated throughout the thickness of the bibulous or non-bibulous material in the test results determination region (for example, 613 that provide specific binding members for one or more drugs, compounds, or metabolites, that can be optionally impregnated in one or more of specified areas, regions, layers, or throughout the thickness of the test strip material in one or more drug, compound, or metabolite preparation, conditioning, assay, or detection zones, and specific binding members for one or more control drugs, compounds, or metabolites can be impregnated in specified areas of the test strip material in one or more reaction or optional control zones). Such impregnation can enhance the extent to which the immobilized reagent can capture a drug, compound, or metabolite present in the migrating sample. Alternatively, reagents, including specific binding members and components of signal producing systems may be applied to specified area or regions of the interior or surface of the bibulous or non-bibulous material. Impregnation of specific binding members into test strip materials or application of specific binding members onto test strip materials may be done manually or by machine.

Nitrocellulose has the advantage that a specific binding member in the test results determination zone can be immobilized without prior chemical treatment. If the porous solid phase material comprises paper, for example, the immobilization of the antibody in the test results determination zone can be performed by chemical coupling using, for example, CNBr, carbonyldiimidazole, or tresyl chloride.

Following the pre-application or migration of a specific binding member and analyte to be detected to the test results determination zones, the remainder of the porous solid phase material can optionally be pre-treated to block any remaining binding sites elsewhere. Blocking can be achieved by pre-treatment with protein (for example bovine serum albumin or milk protein), or with polyvinylalcohol or ethanolamine, or any combination of these agents. A labeled reagent for one or more reagent zones can then be pre-dispensed onto the dry carrier and will become mobile in the carrier when in the moist state. Between each of these various pre-process steps (sensitization, application of unlabeled reagent, blocking and application of labeled reagent), the porous solid phase material should be dried, and then provided as a simple assay where only the sample is added to the application site to provide the test results after allowing time for the dry buffers and/or conditioners to prepare the sample which then runs through the test strip and analyte contained in the sample is then labeled and detectable binding members move to the detection zone and are detected by image collection and analysis of the test strip using a device of non-limiting optional embodiments.

One or more reagents used in the test strip can be pre-applied during manufacturing to the carrier material in a variety of ways. Various “printing” techniques have previously been proposed for application of liquid reagents to carriers, for example micro-syringes, pens using metered pumps, direct printing and ink-jet printing, and any of these techniques can be used in the present context. To facilitate manufacture, the carrier (for example sheet) can be treated with the reagents and then subdivided into smaller portions (for example small narrow strips each embodying the required reagent-containing zones) to provide a plurality of identical carrier units.

In embodiments where the drug, compound, or metabolite is detected by a signal producing system, such as by one or more enzymes that specifically react with the analyte or detectable label, one or more components of the signal producing system can be bound to the drug, compound, or metabolite detection zone of the test strip material in the same manner as specific binding members are bound to the test strip material, as described above. Alternatively or in addition, components of the signal producing system that are included in the sample application zone, the reagent zone, or the drug, compound, or metabolite detection zone of the test strip, or that are included throughout the test strip, may be impregnated into one or more materials of the test strip. This can be achieved either by surface application of solutions of such components or by immersion of the one or more test strip materials into solutions of such components. Components of the signal producing system that are included in the sample application zone, the reagent zone, or the drug, compound, or metabolite detection zone of the test strip, and/or that are included throughout the test strip, may be pre-applied to the surface of one or more test strip materials of the test strip as was described for labeled reagents.

### Sample Application Zone:

One or more sample application zones 610/611/612 is an area of a test strip where a sample, such as a fluid sample, such as a biological fluid sample such as blood, serum, saliva, or urine, or a fluid derived from a biological sample, such as a throat or genital swab, is applied. A sample application zone can include a bibulous or non-bibulous material, such as filter paper, nitrocellulose, glass fibers, polyester or other appropriate materials. One or more materials of the sample application zone may perform a filtering function, such that large particles or cells are prevented from moving through the test strip. One or more sample application zones can be in direct or indirect fluid communication with the remainder of the test strip, including the test results determination zone 613. A direct or indirect fluid communication can be, for example, end-to-end communication, overlap communication, or overlap or end-to-end communication that involves another element, such as a fluid communication structure such as filter paper.

A sample application zone 610/611/612 or the buffer/condition zone 603 can also include compounds or molecules that may be necessary or desirable for optimal performance of the test, for example, buffers or dry buffers, stabilizers, surfactants, salts, reducing agents, or enzymes.

### Reagent Zone:

A test strip can also include one or more reagent zones (603 and/or 607) where reagents useful in the detection of a drug, compound, or metabolite can be provided immobilized (covalent or non-covalent immobilization) or not immobilized. A reagent zone can be on a reagent pad, a separate segment of bibulous or non-bibulous material included on the test strip, or it can be a region of a bibulous or non-bibulous material of a test strip that also includes other zones, such as a drug, compound, or metabolite detection zone. In one aspect of non-limiting optional embodiments, the reagent zone can include a labeled specific binding member, such as antibodies or active fragments thereof attached or linked to a label. Such labeled specific binding members can be made using methods known in the art. A specific binding members can bind a drug, compound, or metabolite and/or can bind an optional compound.

Labels can include, for example, one or more of enzymes, chromophores or fluorophores such as they are known in the art, particularly in immunoassays, or later developed. These reagents are reversibly bound to the reagent zone because they can be mobilized when placed in contact with a fluid, such as a fluid sample passing along a test strip. In another embodiment of non-limiting optional embodiments, the reagent zone can include components of a signal producing system, for example, catalysts, such as enzymes, cofactors, electron donors or acceptors, and/or indicator compounds. A reagent zone can also include compounds or molecules that may be necessary or desirable for optimal performance of the test, for example, buffers (preferably dry buffers or conditioners), stabilizers, surfactants, salts, reducing agents, or enzymes.

### Test Results Determination Zone:

One or more test results determination zones 613, sample preparation zones 603, and/or assay channels 607 can include immobilized or not immobilized reagents that can detect the presence of the drug, compound, or metabolite being tested for, such as but not limited to, drugs of abuse (e.g., illegal, controlled, etc., drug or compound), metabolites, and antibodies. Such reagents are preferably in a dry state and can be covalently immobilized, non-covalently immobilized, or not immobilized in a fluid state. A test result determination zone can include either or both of one or more drug, compound, or metabolite detection zones and one or more control zones.

Depending on the particular format and drug, compound, or metabolite being tested for, a variety of reagents can be provided at the test results determination zone. For example, the test results determination zone can include specific binding members such as antibodies, enzymes, enzymatic substrates, coenzymes, enhancers, second enzymes, activators, cofactors, inhibitors, scavengers, metal ions, and the like. One or more of the reagents provided at the test results determination zone can be bound to the test strip material. Test strips including such reagents are known in the art and can be adapted to the test device of non-limiting optional embodiments.

In a preferred aspect of non-limiting optional embodiments the one or more drug, compound, or metabolite detection zones of the test results determination zone include one or more immobilized (covalently or non-covalently immobilized) specific binding members that bind with one or more drugs, compound, or metabolite of interest, such as one or more drugs, hormones, antibodies, metabolites, or infectious agents, when the drugs, compound, or metabolite are also bound by specific binding members bound to a label as are provided in the reagent zone. Thus, in embodiments where the reagent zone contains one or more specific binding members for the analyte, the specific binding members of the reagent zone and drug, compound, or metabolite detection zone should bind with different epitopes on the drug, compound, or metabolite being tested for. For example, when a labeled specific binding member in the reagent zone binds with the drug, compound, or metabolite, then the immobilized specific binding member in the drug, compound, or metabolite detection zone should bind with another area of drug, compound, or metabolite. Thus, when drug, compound, or metabolite is present in the sample, the drug, compound, or metabolite will bind the labeled anti-drug, compound, or metabolite, which carried along to the test result determination zone at the drug, compound, or metabolite detection zone which binds with the immobilized anti-drug, compound, or metabolite to provide a visual readout.

A drug, compound, or metabolite detection zone can include one or more substrates which change in an optical property (such as color, chemiluminescence or fluorescence) when a drug, compound, or metabolite is present. Such substrates are known in the art, such as, but not limited to, 1,2-phenylenediamine, 5-aminosalicylic acid, 3,3′,5,5′tetra methyl benzidine, or tolidine for peroxidase; 5-bromo-4-chloror-3-indolyl phosphate/nitroblue tetrazolium for alkaline phosphatase and 5-bromo-4-chloro-3-indolyl-beta-D-galactopyranoside, o-nitrophenyl-beta-D-galactopyranoside, napthol-AS-BI-beta-D-galactopyranoside, and 4-methyl-umbelliferyl-beta-D-galactopyranoside for beta galactosidase.

In embodiments where a drug, compound, or metabolite is detected by a signal producing system, one or more components of the signal producing system, such as enzymes, substrates, and/or indicators, can be provided in the drug, compound, or metabolite detection zone. Alternatively, the components of the signal producing system can be provided elsewhere in the test strip and can migrate to the drug, compound, or metabolite detection zone.

Optionally, the test results determination zone can include some form of indicia, such as a marking, indicator, color, and the like for a positive reaction and a marking, indicator for a negative reaction based on the particular format of the assay, e.g., substrates which change in an optical property (such as color, chemiluminescence or fluorescence) when an optional substance is present.

In one aspect of non-limiting optional embodiments, the test strip can include a results determination zone that includes a drug, compound, or metabolite detection zone, and a sample adulteration control zone. A second test strip can include an adulteration control zone. One or more second test strips can be used to detect multiple drugs, compounds or metabolites.

### Orientation of Zones:

One or more various zones of a test strip, including one or more sample application zones, one or more reagent zones, and one or more test result determination zones, including one or more drug, compound, or metabolite detection zones and optionally including one or more control and one or more adulteration zones, can be on a single strip of material, such as filter paper or nitrocellulose, or can be provided on separate pieces of material. A different zones can be made of the same or different material or a combination of materials, but preferably are selected from bibulous materials, such as filter paper, fiberglass mesh and nitrocellulose. A sample application zone preferably includes glass fibers, polyester or filter paper, the one or more reagent zones preferably include glass fibers, polyester or filter paper and the test results determination zone, including one or more drug, compound, or metabolite detection zones and optionally including one or more control, preferably include nitrocellulose.

### Example 2

### Roadside Reader Mobile Device/Application

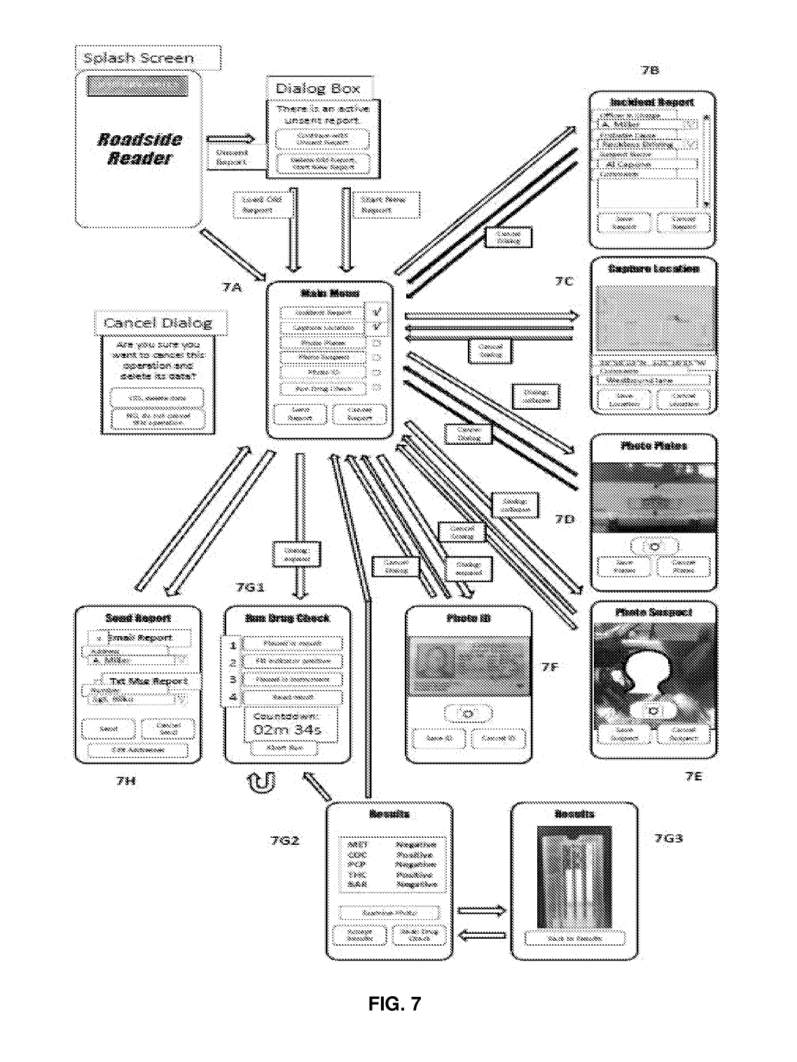

As shown in FIG. 7, non-limiting optional embodiments can include a device and software or firmware, e.g., a mobile app, for use in, e.g., but not limited to a mobile device, e.g., but not limited to, a smart phone (e.g., Android™, iPhone™, Verizon™, Sprint™, T-Mobile™; and the like, made e.g., by Apple™, Samsung™, or the like.), or a tablet or laptop computer (e.g., iPad™, GalaxyIII™, and the like), laptop, netbook, and the like (e.g., Apple™, HP™, Dell™, Toshiba™, and the like), wherein software, such as an mobile app or computer program, can be provided that provide functionality of one or more aspects of non-limiting optional embodiments, e.g., but not limited to, a portable roadside and/or wireless based lateral flow drug test strip systems, devices, or methods, saliva or body fluid conditioning lateral flow test strip systems, devices or methods, optional testing cartridges, systems, and methods, and a method of coupling lateral flow drug test strip systems with test cartridges, a protective case and/or housing, digital camera hardware, computer processor, memory data storage, and software for calibrating, recording, capturing, and analyzing said test strip images to provide drug testing results that are stored in the data memory storage along with ID images and data identifying the person for which saliva or body fluid is collected from, for said drug test, and transmissible via the wireless communications to a remote server or communications device; and/or an optional graphical user interface (GUI) for setting up and conducting the drug testing using the system and for electronically collecting, entering, calibrating, analyzing, and transmitting information, images, data, or results, relating to the drug test or the persons being tested or conducting the test; including electronically providing instructions via said GUI to set up and operate said system; and/or an optional location detection module, wherein the location detection module is a global positioning system (GPS) transceiver; and/or wherein said drug test strip optionally analyzes the presence or amount of a drug, compound, or metabolite thereof in said saliva to determine whether said amount is above a pre-selected threshold or concentration.

As shown in FIG. 7, the mobile device has a user interface that provides one or more of the elements or functions described herein, or as known in the art. As a non-limiting example, as shown in 7A, display or dialog Main Menu (7A) provides a check list of click on functions, e.g., incident report, capture location, photo plates, photo of suspect, photo ID, and drug testing protocol and data collection, as well as sending or cancel report functions. As shown in 7B, the incident report screen can provide information such as officer or person making report, probable cause, suspect name or alias, and further comments. As shown in 7C, the capture location screen can provide address, street, latitude and longitude, and/or other mapping or location information, as well as additional comments. As shown in 7D, the photo plates screen can add, edit or show pictures, diagrams or representations of evidence or other information relevant to the report. As shown in 7E, suspect or witness photos or other image representations can be added, entered, edited, labeled, or removed. As shown in Figure XF, photos or other image representations can be added, edited, labeled or removed relating to identification of suspects or witnesses. As shown in 7G1, drug checking calibrations, set up, protocol steps, timing, sample collection and testing, and results can be entered, done or reported or recorded, e.g., selecting, testing, getting results for and reporting of one or more drug tests, including recording of data, images of test strips, and results, as further shown in 7G2 and 7G3. As shown in 7H, the send report screen can be used to email, text, or otherwise transmit report data or reports to other law enforcement or courts, including any type of data collection or storage, e.g. servers, fax machines, computer data storage, and the like.

### Example 3

### Alternative Test Strip Features and Components

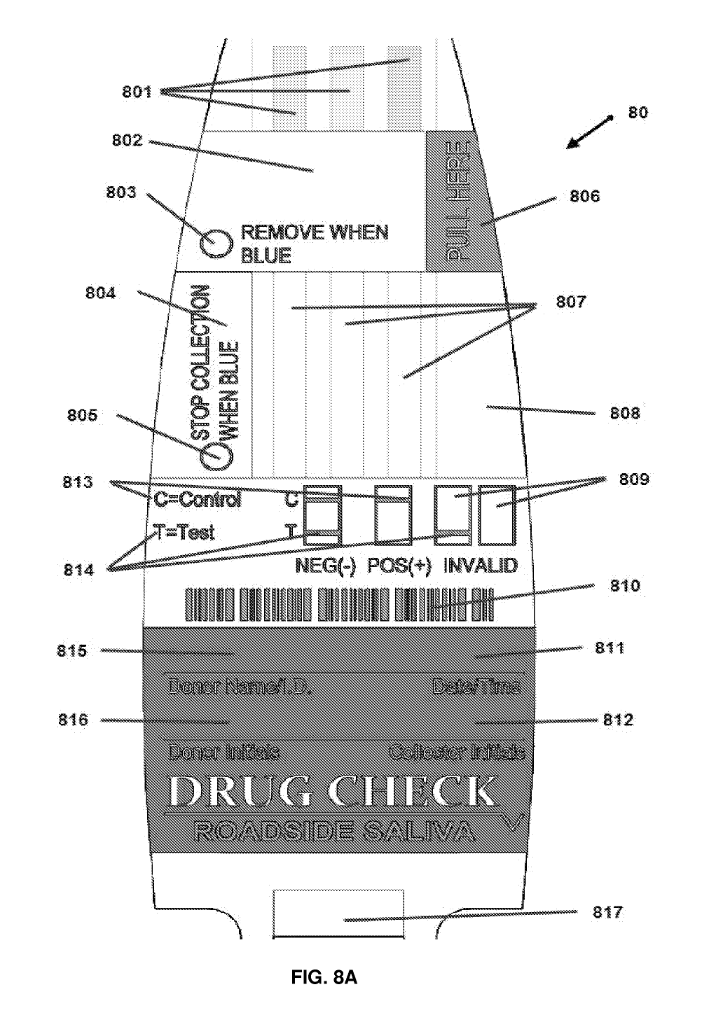

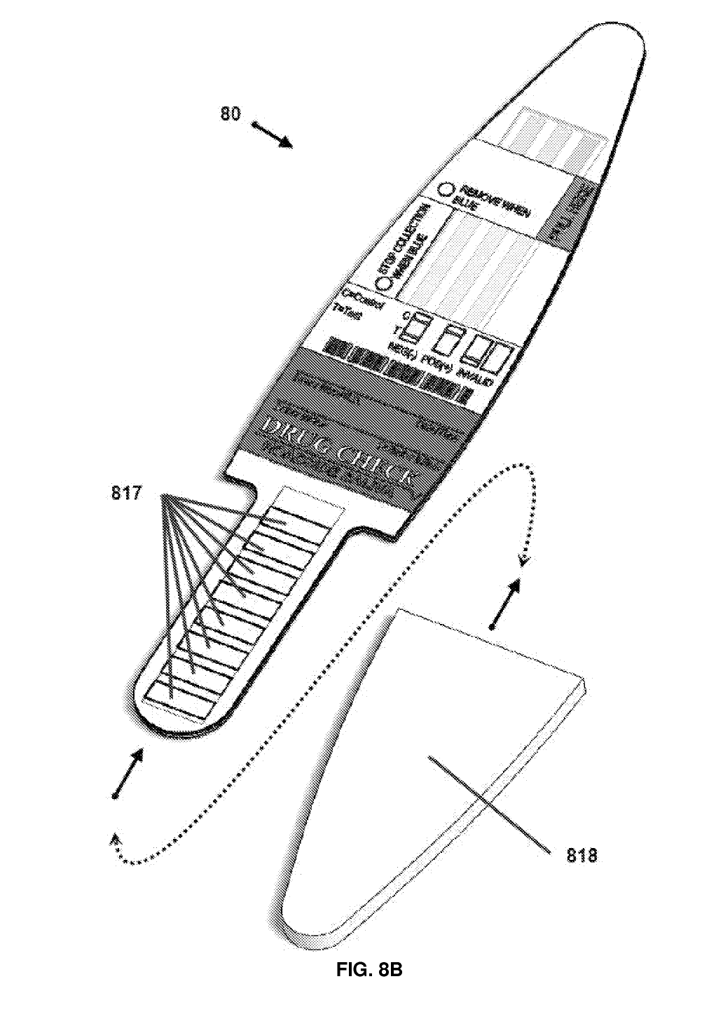

A non-limiting example of a lateral flow test strip of non-limiting optional embodiments is provided with reference to FIGS. 8A-8B.

At least one test strip material can optionally comprise nitrocellulose having a pore size of at least about 1 micron. As shown in a top view of assembled test strip FIG. 8A, a test strip 80 used in context with non-limiting optional embodiments can optionally include one or more of sample application windows 817, assay path channels 807, test result indicator positions 801, result protective cover 802 and pull tab 806, removal indicator 803, stop collection area 804 and stop collection indicator 805, negative and positive control indicators 813, test positive, negative, and invalid indicators 814, and invalid indicators 809, bar code information strip 810, date/time label area 811, collector initials label area 812, donor name/ID label area 815, and/or donor initial label area 816.

As shown in FIG. 8B a removable end protective cover 818 can be provided to do one or more of protect, seal, keep sterilized, prevent moisture or other contamination and the like. Indicia that can include a designation for the test to be performed using the test strip 80. Such indicia may be printed on the test strip material using methods known in the art. Alternatively, indicia may be on other thin members, such as plastic or paper, that are attached to the test strip, such as by adhesives.

A test strip can include one or more materials, as presented above and shown in FIGS. 8A and 8B. If a test strip comprises more than one material, the one or more materials are preferably in fluid communication for movement of the sample through the test strip. One material of a test strip may be overlaid on another material of the test strip, such as for example, filter paper overlaid on nitrocellulose. Alternatively or in addition, a test strip may include a region comprising one or more materials followed by a region comprising one or more different materials. In this case, the regions are in fluid communication and may or may not partially overlap one another.

A material or materials of the test strip can be bound to a support or solid surface such as shown in FIGS. 8A-8B, for example, and may have an absorbent pad either as an integral part or in liquid contact. For example, as shown in FIGS. 8A and 8B, a test strip may comprise nitrocellulose sheet “backed”, for example with a supporting sheet, such as a plastic sheet, to increase its handling strength. This can be manufactured by forming a thin layer of nitrocellulose on a sheet of backing material or by a pressing machine or device, such as a high speed press. A actual pore size of the nitrocellulose when backed in this manner will tend to be lower than that of the corresponding unbacked material. Alternatively, a pre-formed sheet of nitrocellulose and/or one or more other bibulous or non-bibulous materials can be attached to at least one supporting sheet, such as a sheet made of polymers (see, e.g., U.S. Pat. No. 5,656,503, entirely incorporated by reference). A cover, intermediate and/or supporting sheet can be transparent, translucent or opaque, and/or provided with windows, gaps or spaces. In the aspect of non-limiting optional embodiments where the support sheet is transparent, the supporting sheet is preferably moisture impervious but can be moisture resistant or moisture pervious. In another embodiment of non-limiting optional embodiments the test strip can be viewed through a window comprised of a transparent material such as glass, plastic, or mylar, but preferably break resistant.

In the following discussion strips of test strip material will be described by way of illustration and not limitation, e.g., as shown in FIGS. 8A and 8B.

Generally, test strips of a test device of non-limiting optional embodiments include one or more sample application zones (817) and a test results determination region (801). A test results determination region can include either or both of one of more drug, compound, or metabolite detection zones (e.g., 801), that can exclude or include additional or integrated optional control zones. Optionally, a test strip can include a reagent zone, e.g., a buffer, (e.g., a dry buffer) or conditioning compounds zone.

One or more specific binding members in the test results determination region of the test strip can be impregnated throughout the thickness of the bibulous or non-bibulous material in the test results determination region (for example, that provide specific binding members for one or more drugs, compounds, or metabolites, that can be optionally impregnated in one or more of specified areas, regions, layers, or throughout the thickness of the test strip material in one or more drug, compound, or metabolite preparation, conditioning, assay, or detection zones, and specific binding members for one or more control drugs, compounds, or metabolites can be impregnated in specified areas of the test strip material in one or more reaction or optional control zones). Such impregnation can enhance the extent to which the immobilized reagent can capture a drug, compound, or metabolite present in the migrating sample. Alternatively, reagents, including specific binding members and components of signal producing systems may be applied to specified area or regions of the interior or surface of the bibulous or non-bibulous material. Impregnation of specific binding members into test strip materials or application of specific binding members onto test strip materials may be done manually or by machine.

Following the pre-application or migration of a specific binding member and analyte to be detected to the test results determination zone, the remainder of the porous solid phase material can optionally be pre-treated to block any remaining binding sites elsewhere. Blocking can be achieved by pre-treatment with protein (for example bovine serum albumin or milk protein), or with polyvinylalcohol or ethanolamine, or any combination of these agents. A labeled reagent for the reagent zone can then be pre-dispensed onto the dry carrier and will become mobile in the carrier when in the moist state. Between each of these various pre-process steps (sensitization, application of unlabeled reagent, blocking and application of labeled reagent), the porous solid phase material should be dried, and then provided as a simple assay where only the sample is added to the application site to provide the test results after allowing time for the dry buffers and/or conditioners to prepare the sample which then runs through the test strip and analyte contained in the sample is then labeled and detectable binding members move to the detection zone and are detected by image collection and analysis of the test strip using a device of non-limiting optional embodiments.

One or more reagents used in the test strip can be pre-applied during manufacturing to the carrier material in a variety of ways. Various “printing” techniques have previously been proposed for application of liquid reagents to carriers, for example micro-syringes, pens using metered pumps, direct printing and ink-jet printing, and any of these techniques can be used in the present context. To facilitate manufacture, the carrier (for example sheet) can be treated with the reagents and then subdivided into smaller portions (for example small narrow strips each embodying the required reagent-containing zones) to provide a plurality of identical carrier units.

In embodiments where the drug, compound, or metabolite is detected by a signal producing system, such as by one or more enzymes that specifically react with the analyte, one or more components of the signal producing system can be bound to the drug, compound, or metabolite detection zone of the test strip material in the same manner as specific binding members are bound to the test strip material, as described above. Alternatively or in addition, components of the signal producing system that are included in the sample application zone, the reagent zone, or the drug, compound, or metabolite detection zone of the test strip, or that are included throughout the test strip, may be impregnated into one or more materials of the test strip. This can be achieved either by surface application of solutions of such components or by immersion of the one or more test strip materials into solutions of such components. Components of the signal producing system that are included in the sample application zone, the reagent zone, or the drug, compound, or metabolite detection zone of the test strip, and/or that are included throughout the test strip, may be pre-applied to the surface of one or more test strip materials of the test strip as was described for labeled reagents.

### Sample Application Zone:

Sample application zones in 817 of a test strip are provided where a sample, such as a fluid sample, such as a biological fluid sample such as blood, serum, saliva, or urine, or a fluid derived from a biological sample, such as a throat or genital swab, is applied. A sample application zone can include a bibulous or non-bibulous material, such as filter paper, nitrocellulose, glass fibers, polyester or other appropriate materials. One or more materials of the sample application zone may perform a filtering function, such that large particles or cells are prevented from moving through the test strip. A sample application zone can be in direct or indirect fluid communication with the remainder of the test strip, including the test results determination zone 801. A direct or indirect fluid communication can be, for example, end-to-end communication, overlap communication, or overlap or end-to-end communication that involves another element, such as a fluid communication structure such as filter paper.

Sample application zones 817 and/or the buffer/condition zone can also include compounds or molecules that may be necessary or desirable for optimal performance of the test, for example, buffers or dry buffers, stabilizers, surfactants, salts, reducing agents, or enzymes.

### Reagent Zone:

A test strip can also include one or more reagent zones where reagents useful in the detection of a drug, compound, or metabolite can be provided immobilized (covalent or non-covalent immobilization) or not immobilized. A reagent zone can be on a reagent pad, a separate segment of bibulous or non-bibulous material included on the test strip, or it can be a region of a bibulous or non-bibulous material of a test strip that also includes other zones, such as a drug, compound, or metabolite detection zone. In one aspect of non-limiting optional embodiments, the reagent zone can include a labeled specific binding member, such as antibodies or active fragments thereof attached or linked to a label. Such labeled specific binding members can be made using methods known in the art. A specific binding members can bind a drug, compound, or metabolite and/or can bind an optional compound.

Labels can include, for example, enzymes, chromophores or fluorophores such as they are known in the art, particularly in immunoassays, or later developed. These reagents are reversibly bound to the reagent zone because they can be mobilized when placed in contact with a fluid, such as a fluid sample passing along a test strip. In another embodiment of non-limiting optional embodiments, the reagent zone can include components of a signal producing system, for example, catalysts, such as enzymes, cofactors, electron donors or acceptors, and/or indicator compounds. A reagent zone can also include compounds or molecules that may be necessary or desirable for optimal performance of the test, for example, buffers (preferably dry buffers or conditioners), stabilizers, surfactants, salts, reducing agents, or enzymes.

### Test Results Determination Zone:

One or more test results determination zones 801, sample preparation zones, and/or assay channels 807 can include immobilized or not immobilized reagents that can detect the presence of the drug, compound, or metabolite being tested for, such as but not limited to, drugs of abuse (e.g., illegal, controlled, etc., drug or compound), metabolites, and antibodies. Such reagents are preferably in a dry state and can be covalently immobilized, non-covalently immobilized, or not immobilized in a fluid state. Test result determination zones can include either or both of one or more drug, compound, or metabolite detection zones and one or more control zones.

Depending on the particular format and drug, compound, or metabolite being tested for, a variety of reagents can be provided at the test results determination zone. For example, the test results determination zone can include specific binding members such as antibodies, enzymes, enzymatic substrates, coenzymes, enhancers, second enzymes, activators, cofactors, inhibitors, scavengers, metal ions, and the like. One or more of the reagents provided at the test results determination zone can be bound to the test strip material. Test strips including such reagents are known in the art and can be adapted to the test device of non-limiting optional embodiments.

In a preferred aspect of non-limiting optional embodiments the one or more drug, compound, or metabolite detection zones of the test results determination zone include one or more immobilized (covalently or non-covalently immobilized) specific binding members that bind with one or more drugs, compound, or metabolite of interest, such as one or more drugs, hormones, antibodies, metabolites, or infectious agents, when the drugs, compound, or metabolite are also bound by specific binding members bound to a label as are provided in the reagent zone. Thus, in embodiments where the reagent zone contains one or more specific binding members for the analyte, the specific binding members of the reagent zone and drug, compound, or metabolite detection zone should bind with different epitopes on the drug, compound, or metabolite being tested for. For example, when a labeled specific binding member in the reagent zone binds with the drug, compound, or metabolite, then the immobilized specific binding member in the drug, compound, or metabolite detection zone should bind with another area of drug, compound, or metabolite. Thus, when drug, compound, or metabolite is present in the sample, the drug, compound, or metabolite will bind the labeled anti-drug, compound, or metabolite, which carried along to the test result determination zone at the drug, compound, or metabolite detection zone which binds with the immobilized anti-drug, compound, or metabolite to provide a visual readout.

A drug, compound, or metabolite detection zone can include substrates which change in an optical property (such as color, chemiluminescence or fluorescence) when a drug, compound, or metabolite is present. Such substrates are known in the art, such as, but not limited to, 1,2-phenylenediamine, 5-aminosalicylic acid, 3,3′,5,5′tetra methyl benzidine, or tolidine for peroxidase; 5-bromo-4-chloror-3-indolyl phosphate/nitroblue tetrazolium for alkaline phosphatase and 5-bromo-4-chloro-3-indolyl-beta-D-galactopyranoside, o-nitrophenyl-beta-D-galactopyranoside, napthol-AS-BI-beta-D-galactopyranoside, and 4-methyl-umbelliferyl-beta-D-galactopyranoside for beta galactosidase.

In embodiments where a drug, compound, or metabolite is detected by a signal producing system, one or more components of the signal producing system, such as enzymes, substrates, and/or indicators, can be provided in the drug, compound, or metabolite detection zone. Alternatively, the components of the signal producing system can be provided elsewhere in the test strip and can migrate to the drug, compound, or metabolite detection zone.

Optionally, the test results determination zone can include some form of indicia, such as a marking, indicator, color, and the like for a positive reaction and a marking, indicator for a negative reaction based on the particular format of the assay, e.g., substrates which change in an optical property (such as color, chemiluminescence or fluorescence) when an optional substance is present.

In one aspect of non-limiting optional embodiments, the test strip can include a results determination zone that includes a drug, compound, or metabolite detection zone, and a sample adulteration control zone. A second test strip can include an adulteration control zone. One or more second test strips can be used to detect multiple drugs, compounds or metabolites.

### Orientation of Zones:

A various zones of a test strip, including a sample application zone, one or more reagent zones, and one or more test result determination zones, including one or more drug, compound, or metabolite detection zones and optionally including one or more control and one or more adulteration zones, can be on a single strip of material, such as filter paper or nitrocellulose, or can be provided on separate pieces of material. A different zones can be made of the same or different material or a combination of materials, but preferably are selected from bibulous materials, such as filter paper, fiberglass mesh and nitrocellulose. A sample application zone preferably includes glass fibers, polyester or filter paper, the one or more reagent zones preferably include glass fibers, polyester or filter paper and the test results determination zone, including one or more drug, compound, or metabolite detection zones and optionally including one or more control, preferably include nitrocellulose.

The above disclosed subject matter is to be considered illustrative, and not restrictive, and the appended claims are intended to cover all such modifications, enhancements, and other embodiments which fall within the true spirit and scope of the present invention. Thus, to the maximum extent allowed by law, the scope of non-limiting optional embodiments is to be determined by the broadest permissible interpretation of the following claims and their equivalents, and shall not be restricted or limited by the foregoing detailed description.

What is claimed is:

1. A portable lateral flow drug test strip system for testing the presence or amount of a drug, compound, or metabolite thereof, comprising:
an exterior protective case;
a test cartridge receptor provided within said case to receive a test cartridge having one or more reading bays configured to hold pre-calibrated lateral flow drug test strips inserted therein, said test cartridge or test strip comprising or provided with soluble or dry buffers for preparing a saliva sample from a person being drug tested that is applied to said one or more drug test strips,
a housing provided with or in the protective case for positioning digital camera hardware, illumination hardware, a computer processor, data memory storage, and wireless communications, the digital camera hardware comprising imaging optics to produce a digital image of one or more of said test strips in said test cartridge and related testing and identification images and data;
software provided in said data memory storage and loadable onto said computer processor, said software calibrating, recording, capturing, and analyzing said test strip images to provide drug testing results that are stored in said data memory storage along with ID images and data identifying the person for which saliva is collected from, said results, ID images and data transmissible via said wireless communications to a remote server or communications device; and,
a graphical user interface (GUI) for providing tester controlling and using the system for electronically collecting, entering, calibrating, analyzing, and transmitting information, images, data, or results, relating to the drug test or the persons being tested or conducting the test; including electronically providing instructions via said GUI to set up and operate said system; and
a location detection module, wherein the location detection module is a global positioning system (GPS) transceiver;
wherein said drug test strip or a casing thereof when provided with the conditioned sample indicates the presence or amount of a drug, compound, or metabolite thereof in said saliva to determine whether said amount is above a pre-selected threshold or concentration.

2. A portable lateral flow drug test strip system of claim 1, wherein the system comprises a smart phone comprising three or more selected from the group consisting of said: digital camera hardware, illumination hardware, computer processor, data memory storage, wireless communications, imaging optics, software, GUI, and global positioning system (GPS) transceiver or other location detection module.

3. A portable lateral flow drug test strip system of claim 1, wherein the drug test image indicates the presence or amount of drug specific to each drug test strip.

4. A portable lateral flow drug test strip system of claim 1, further comprising a heater to maintain said drug test strips within specified temperature ranges suitable for said drug test.

5. A portable lateral flow drug test strip system of claim 1, further comprising illumination hardware for illuminating the test strip for digital image collection.

6. A portable lateral flow drug test strip system of claim 1, wherein said reading bays comprise at least a first type of reading bay having a first width and a first depth and the second type of reading bay has a second width and a second depth, and wherein each of said reading bays has a single opening to receive each of the first type of test cartridge and the second type of test cartridge without the use of a separate adapter.

7. A portable lateral flow drug test strip system of claim 1, wherein the test cartridge receptor includes an internal track guide for positioned insertion of test cartridges sized or calibrated to pre-position said test strips relative to said digital camera hardware to size the digital image for said electronically collecting, entering, calibrating, analyzing, and transmitting information, images, data, or results, relating to the drug test.

8. A portable lateral flow drug test strip system of claim 1, wherein the computer and/or software comprises a test cartridge or test strip detection and identification logic to detect and identify the presence of a particular test cartridge or test strip to determine the type of drug test to provide said software calibrating, recording, capturing, and analyzing said test strip images to provide drug testing results that are stored in said data memory storage.

9. A portable lateral flow drug test strip system of claim 1, wherein the test cartridge receptor has a side, bottom or top insertion track or alignment for receiving said test cartridges.

10. A portable lateral flow drug test strip system of claim 1, wherein the location detection module provides location data that is communicated to the computer processor or memory data storage to record the location of the drug test performed using said wireless based lateral flow drug test strip system.

11. A portable lateral flow drug test strip system of claim 1, further comprising an environmental sensor to detect environmental conditions.

12. A portable lateral flow drug test strip system of claim 11, wherein the environmental conditions are selected from humidity and temperature data, airborne contaminants, and toxins.

13. A portable lateral flow drug test strip system of claim 1, wherein said pre-selected threshold is the legal limit for said drug, compound, or metabolite thereof, or said preselected concentration is the legal or illegal concentration of said drug, compound, or metabolite thereof, for the corresponding legal jurisdiction.

14. A portable lateral flow drug test strip system of claim 1, wherein said drug or compound is selected from alcohol, cocaine, methamphetamine, heroin, THC, PCP, psilocybin, opiate drugs, or derivatives thereof.

15. A portable lateral flow drug test strip system of claim 1, wherein the person performing the test (tester) performs at least one selected from the group consisting of removing the test device from the packaging, removing a cap from an oral fluid collecting device or placing the oral fluid collecting device in a test subject's mouth.

16. A portable lateral flow drug test strip system of claim 15, wherein the tester opens the case on the electronic device and/or via the GUI in order to initiate the drug test.

17. A portable lateral flow drug test strip system of claim 15, wherein the tester collects oral fluid until sufficient oral fluid has been collected (absorbed) by the test device as indicated by a specified color changing fill indicator or the tester removes from the mouth of the test subject when complete or replaces the cap.

18. A portable lateral flow drug test strip system of claim 15, wherein the tester removes a test initiation sticker or inserts the collected oral fluid into test cartridge in the system.

19. A portable lateral flow drug test strip system of claim 18, wherein the test timing is automatically initiated by insertion of collected oral fluid, or the tester initiates test timing on the GUI of the device after insertion.

20. A portable lateral flow drug test strip system of claim 15, wherein the digital camera hardware takes an initial picture of the test device to determine the specific test type information or lot numbers or any other specific information.

21. A portable lateral flow drug test strip system of claim 15, wherein the case initiates internal lighting automatically to illuminate the test strips when an image is being captured by the digital camera hardware.

22. A portable lateral flow drug test strip system of claim 1, wherein test or control line intensities on each test strip tested are electronically determined from the digital image by said system.

23. A portable lateral flow drug test strip system of claim 22, wherein the positive or negative result, or validity, of the test is electronically determined by analyzing said control line intensities for each test strip.

24. A portable lateral flow drug test strip system of claim 15, wherein the results of the test are electronically communicated to at least one of a tester, law enforcement personnel, or a court.

25. A method for portable lateral flow drug test strip testing the presence or amount of a drug, compound, or metabolite thereof, the method comprising the steps of:
(a) providing a saliva sample from a person being drug tested;
(b) providing a potable lateral flow drug test strip system comprising
(i) an exterior protective case;
(ii) a test cartridge receptor provided within said case to receive a test cartridge having one or more reading bays configured to hold pre-calibrated lateral flow drug test strips inserted therein, said test cartridge or test strip comprising soluble or dry buffers for preparing a saliva sample from a person being drug tested that is applied to said one or more drug test strips;
(iii) a housing provided with or in the protective case for positioning:
(iv) digital camera hardware comprising imaging optics to produce a digital image of one or more of said test strips in said test cartridge and related testing and identification images and data;
(v) illumination hardware;
(vi) computer processor;
(vii) data memory storage; and
(viii) wireless communications;
(ix) software, provided in said data memory storage and loadable onto said computer processor, said software calibrating, recording, capturing, and analyzing said test strip images to provide drug testing results that are stored in said data memory storage along with ID images and data identifying the person for which saliva is collected from, for said drug test and transmissible via said wireless communications to a remote server or communications device; and a graphical user interface (GUI) for setting up and conducting the drug testing using the system and for electronically collecting, entering, calibrating, analyzing, and transmitting information, images, data, or results, relating to the drug test or the persons being tested or conducting the test; including electronically providing instructions via said GUI to set up and operate said system; and a location detection module, wherein the location detection module is a global positioning system (GPS) transceiver or other location determination service;
(c) conditioning said saliva sample using said dry buffer or conditioning media to provide conditioned sample;
(d) applying the conditioned saliva sample to said test strip or cartridge to provide a sampled test strip;
(e) inserting the sampled test strip into said cartridge or cartridge/test strip receptor in said case;
(f) using said software and computer processor via said GUI, electronically calibrating, recording, capturing, and analyzing said test strip images to provide drug testing results that are stored in said data memory storage along with ID images and data identifying the person for which said saliva sample is collected from, for said drug test, to determine the presence or amount of a drug, compound, or metabolite thereof, in said saliva to, in order to determine whether said amount is above a pre-selected threshold or concentration.

26. A method of claim 25, wherein a smart phone is used comprising three or more selected from the group consisting of said: digital camera hardware, illumination hardware, computer processor, data memory storage, wireless communications, imaging optics, software, GUI, and global positioning system (GPS) transceiver.

27. A method of claim 25, wherein the drug test image indicates the presence or amount of drug specific to each drug test strip.

28. A method of claim 25, further comprising heating said drug test strips within specified temperature ranges suitable for said drug test.

29. A method of claim 25, further comprising illuminating the test strip for digital image collection.
